(19) United States
(12) Patent Application Publication
(10) Pub. No.: US 2012/0258539 A1
(43) Pub. Date: Oct. 11, 2012

(54) USE OF NIBP POLYPEPTIDES

(75) Inventor: Wenhui Hu, Cherry Hill, NJ (US)

(73) Assignee: Temple University of the Commonwealth of Higher Education Office of Technology Transfer, Philadelphia, PA (US)

(21) Appl. No.: 13/499,932

(22) PCT Filed: Oct. 12, 2010
(86) PCT No.: PCT/US10/52302

§ 371 Date: Apr. 3, 2012

Related U.S. Application Data
(60) Provisional application No. 61/251,013, filed on Oct. 13, 2009.

(51) Int. Cl. C12N 15/85; C12N 5/10

(52) U.S. Cl. 435/455; 435/325

(57) ABSTRACT

Methods for regulating NF-κB activation in cells comprising introducing into the cell a vector comprising a nucleic acid sequence encoding a NIK and IKK2 Binding Protein (NIBP) polypeptide, wherein the NIBP polypeptide is expressed in the cell, are provided. Also provided are methods for reversing the cancerous phenotype of a cancer cell and for modulating neuronal differentiation.

### CROSS REFERENCE TO RELATED APPLICATIONS

This application claims priority to U.S. Provisional Application No. 61/251,013, filed Oct. 13, 2009, and the contents of which are incorporated by reference herein in their entireties and for all purposes.

### FIELD OF THE INVENTION

This invention relates generally to the fields of cell and molecular biology, neuroscience, immunology and gene therapy. More specifically, the invention relates to the use of NIK and IKK2 Binding Protein (NIBP) polypeptides for regulating (e.g., inhibiting) NF-κB activation in a cell, reversing the cancerous phenotype of a cancer cell, and modulating neural differentiation.

### BACKGROUND OF THE INVENTION

Nuclear factor κB (NF-κB) plays a pivotal role in many biological processes (such as inflammation, immunity, stress response, neural plasticity) and pathophysiologic disorders (such as cancer, inflammatory diseases, autoimmune diseases and neurodegenerative diseases) (Boyce et al., 2010; Hacker and Karin, 2006; Lin et al., 2010; Mancino and Lawrence, 2010; O'Sullivan et al., 2010; Perkins, 2007; Wong and Tergaonkar, 2009).

In many types of human tumors, especially breast cancer, constitutive elevation of NF-κB activity and its signaling has been reported (Biswas et al., 2004; Cao and Karin, 2003; Jackson-Bernitsas et al., 2006; Karin, 2006; Karin and Greten, 2005; Pacifico and Leonardi, 2006; Romieu-Mourez et al., 2001). However, the origins and mechanisms of constitutive NF-κB activation remain unclear (Bhat-Nakshatri et al., 2002; Eddy et al., 2005). Infection and inflammation are known to affect cancer development and progression (Karin and Greten, 2005). Sustained activation of NF-κB is a critical mediator for inflammation-linked cancer (Greten et al., 2004; Karin, 2006; Mantovani and Balkwill, 2006). NF-κB-regulated immune responses are closely related to cancer development. NF-κB activation is also linked to drug-resistance (Ahmed et al., 2006; Montagut et al., 2006) and poor prognosis (Wang et al., 2005). In various types of chronic diseases, NF-κB is also continuously activated.

NF-κB is activated by various stimuli and regulates a large number of genes involved in oncogenesis and inflammatory responses. A canonical pathway and an alternative pathway for NF-κB activation have been identified (Bonizzi and Karin, 2004). The canonical pathway triggered by stimuli such as TNFα and IL-1β depends on the signalsome of IκB kinase (IKK), which consists of at least two catalytic subunits (IKK1 and IKK2) and a regulatory subunit (IKKγ). The IKK phosphorylates the inhibitor proteins of NF-κB (IκBs) to induce their ubiquitination and degradation, resulting in the nuclear translocation of NF-κB dimers (mainly p65/p50) and the activation of target genes. The alternative pathway relies on the phosphorylation of IKK1 by NF-κB inducing kinase (NIK) to induce p100 processing into p52 and the nuclear translocation of ReIB/p52 dimers.

High constitutive expression of IKK is present in breast cancer but not normal cells (Biswas et al., 2004; Buchholz et al., 2005; Cogswell et al., 2000; Dejardin et al., 1995; Karin, 2006; Kim et al., 2000; Nakshatri et al., 1997; Pacifico and Leonardi, 2006; Patel et al., 2000; Romieu-Mourez et al., 2001; Sovak et al., 1997). IKK2 is critical in cancer metastasis (Huber et al., 2004; Park et al., 2007) and tumorigenesis (Greten et al., 2004; Hu and Hung, 2005). In breast cancers, IKK2 overexpression is associated with cytoplasmic accumulation of p21, an antiapoptotic factor involved in tumorigenesis (Ping et al., 2006). IKK2 induces degradation of IκB, leading to constitutive survival signaling (Hu et al., 2004). IKK2-specific inhibitors have been targeted for therapeutic development (Ciucci et al., 2006; Frelin et al., 2005; Haffner et al., 2006; Kim et al., 2006; Luo et al., 2005; Ruocco and Karin, 2005; Tanaka et al., 2006). However, the specificity of NFκB signaling and the regulatory mechanisms for IKK2/NFκB activation remain elusive. NIK is also upregulated in breast cancer and contributes to constitutive NFκB activation (Yamaguchi et al., 2009).

A number of clinical findings have identified the importance of NIBP disruption in neurodevelopmental disorders and other brain diseases. Homozygous NIBP non-sense mutation is closely correlated with autosomal recessive mental retardation and neonatal microcephaly (Mir et al., 2009; Mochida et al., 2009; Philippe et al., 2009). Homozygous deletion of the entire NIBP gene leads to severe developmental delay, retinal dystrophy and hearing loss (Koifman et al., 2010). Heterozygous deletion of NIBP partial genome (containing exon 1-15) leads to maternal autism (Riendeau, 2009). Two SNPs (single nucleotide polymorphisms) in NIBP that contribute to maternal effects on human height (Kent et al., 2009) and one SNP associated with the prevalence of stroke (Yoshida et al., 2010) have been identified in a genome-wide assay. Several cases of patients with different types of cancers have also been reported to be correlated with NIBP (Ghobrial et al., 2010; Kim et al., 2008; Ross et al., 2007).

It has been showed that NIBP interacts with NIK and IKK2 (Hu et al., 2005). Recent studies demonstrated that NIBP, as an essential member of endoplasmic reticulum (ER)-Golgi trafficking complex TRAPP (Transport protein particle), interacts with other members of TRAPP such as Bet3 (Kummel et al., 2008) and Trs33 (Tokarev et al., 2009), implying that NIBP may regulate the trafficking or transport processes. A new study using yeast two-hybrid screening identified a novel partner of NIBP, the nonstructural protein 5A (NS5A) from bovine viral diarrhea virus (BVDV) (Zahoor et al., 2010). BVDV NS5a shares many features with its counterpart NS5A from hepatitis C virus (HCV). The interaction of NIBP with NS5A inhibits the replication of BVDV and potentially HCV because NIBP knockdown enhances viral RNA replication.

There exists a need for methods of treating diseases and disorders in various areas, including neurodevelopment, neurodegenesis, tumorigenesis and virus defense, by regulating NF-κB activation via NIBP polypeptide.

### SUMMARY OF THE INVENTION

The invention features methods for inhibiting NF-κB activation in a cell. The methods generally comprise introducing into the cell a vector comprising a nucleic acid sequence encoding a NIK and IKK2 Binding Protein (NIBP) polypeptide, wherein the NIBP polypeptide is expressed in the cell. The NIBP polypeptide may have an amino acid sequence selected from the group consisting of SEQ ID NOs: 3 and 5-7. The NF-κB activation in the cell may be constitutive. It may also be stimulated or induced by, for example, TNFα and IL-1β. The cell can be any cell, preferably a cancer cell. Exemplary cancer cells include breast, gut, liver, colorectal, cervix, prostate, lung and brain cancer cells.

The invention also features methods for reversing the cancerous phenotype of a cancer cell. In general, the methods comprise introducing into the cell a vector comprising a nucleic acid sequence encoding a NIK and IKK2 Binding Protein (NIBP) polypeptide, wherein the NIBP polypeptide is expressed in the cell. The NIBP polypeptide may have an amino acid sequence selected from the group consisting of SEQ ID NOs: 3-7 and 13-16. Examples of reversing the cancerous phenotype include inhibiting cell proliferation and inducing cell death. The cancer cell may be a breast, gut, liver, colorectal, cervix, prostate, lung and brain cancer cell.

The invention further features methods for modulating neuronal differentiation of a cell. The methods comprise introducing into the cell a vector comprising a nucleic acid sequence encoding a NIK and IKK2 Binding Protein (NIBP) polypeptide, wherein the NIBP polypeptide is expressed in the cell. Preferably, the cell is selected from the group consisting of a neural stem cell (NSC), a neural progenitor cell (NPC), and a cell having disrupted expression of the NIBP.

Also featured are host cells comprising a vector. The vector comprises a nucleic acid sequence encoding a NIK and IKK2 Binding Protein (NIBP) polypeptide, and the host cells are capable of expressing the NIBP polypeptide. The NIBP polypeptide may have an amino acid sequence selected from the group consisting of SEQ ID NOs: 1-7 and 13-16.

### BRIEF DESCRIPTION OF THE DRAWINGS

FIGS. 1(A) and 1(B) show constitutive expression of NIBP mRNA in cancer cell lines by Northern blot (A) and Real-time PCR using two pairs of NIBP primers (B). Data show the absolute value after GAPDH normalization using NIBP plasmid as a standard.

FIG. 2 shows immunofluorescent staining of a frozen tissue array from human normal (top panels) and tumor (bottom panels) using affinity-purified anti-NIBP polyclonal antibodies. Intensive immunoreactivity is present in breast invasive ductal carcinoma (left panels) and hepatocellular carcinoma (right panels).

FIG. 3 shows NIBP primer 2 (771-914) real-time PCR analysis of a human cancer survey tissue cDNA panel demonstrating a dramatic increase in tumors from breast, kidney, and liver. Data represent relative fold-change to the healthy control sample after beta-actin normalization.

FIGS. 4(A)-(C) show interaction of NIBP and IKK2 in MCF7 cells. Whole cell lysates were immunoprecipitated with anti-NIBP antibody or control IgG followed by immunoblotting with antibodies against IKK1/2 (A, C) or phosphorylated IKK1/2 (B).

FIG. 5 shows overexpression of NIBP(960) in MDA-MB-231 cells infected by lentivirus (LV). After 2 passages, the cells were infected by Adenovirus carrying NF-κB-luciferase for 2 days and treated with TNFα 10 ng/ml overnight.

FIGS. 6(A) and 6(B) show some NIBP isoforms in Genbank database (A), and a diagram of NIBP mutants and submutants used in connection with the invention (B).

FIGS. 7(A) and 7(B) show the interaction of NIBP mutants with NIK (A) and IKK2 (B). HEK293T cells were transfected with indicated vectors. After 24 h, lysate (Lys) was immunoprecipitated with anti-Flag or IgG control antibody followed by immunoblotting (IB) with anti-Myc antibody. The expression of Flag-NIBP mutants in the immunoprecipitated complex was verified by immunoblotting with anti-Flag antibody.

FIG. 8 shows that subdomain (121-211) of NIBP-mutF interacts with NIK. HEK293T cells were transfected with the indicated vectors. After 24 h, lysate (Lys) was immunoprecipitated with anti-Flag antibody followed by immunoblotting (IB) with anti-Myc antibody. The expression of Flag-fusion protein was verified by immunocytochemistry (ICC).

FIG. 9 shows that subdomains of NIBP-mutE interact with NIK and not IKK2. Lysate (Lys) was used to confirm the expression of NIK and IKK2. The mutant expression was verified by immunocytochemistry (not shown).

FIG. 10 shows that NIBP interacts with the N-terminus of IKK2. HEK293T cells were co-transfected with pRK-Myc-IKK2(1-103) and pRK-Flag-NIBP(960). After 24 h, protein extracts were immunoprecipitated with anti-Flag antibody. Co-immunoprecipitated Myc-IKK2(1-103) was detected by Western blot with anti-Myc antibody. The expression of both fusion proteins was confirmed by Western blot in the lysate (Lys).

FIG. 11 shows the effect of various isoforms on NF-κB activation. MDA-MB-231 cells were co-transfected by TurboFectin8.0 with empty pRK-Flag vector or various isoforms of NIBP with NF-κB-SEAP reporter and pcDNA3-luciferase for 2 days. Data represent relative fold-changes compared with empty-vector controls.

FIG. 12 shows that NIBP120 inhibits constitutive and TNFα-stimulated and IKK2-mediated NF-κB activation. HEK293T cells were co-transfected with the indicated vectors and NF-κB firefly-luciferase and pcDNA3-renilla-luciferase reporter vectors. After 24 h, cells were treated with or without TNFα (10 ng/ml) for 24 h before dual luciferase assay. Data are expressed as relative change compared with empty vector control.

FIG. 13 shows NIBP120 inhibition of TNFα-induced phosphorylation of IKK1/2. MDA-MB-231 cells at 60% confluence in 6-well plates were transfected with empty vector or NIBP mutant vectors. After 5 days, cells were treated with TNFα for the indicated times and Western blotting was performed with the indicated antibodies in the same blot after stripping.

FIG. 14 shows NIBP120 enhanced constitutive and TNFα-stimulated phosphorylation of JNK, ERK1/2 and p38 MAPK. MDA-MB-231 cells at 60% confluence in 6-well plates were transfected with an empty vector or NIBP120 vector. After 5 days, cells were treated with TNFα for the indicated time and Western blotting was performed with the indicated antibodies in the same blot after stripping.

FIGS. 15(A) and 15(B) show submutants of NIBP120 (mutE) inhibited constitutive and TNFα-stimulated NF-κB activation. HEK293T cells (A) or breast cancer cell lines (B) were co-transfected with an empty vector or NIBP120 submutants plus NF-κB-firefly luciferase (A) or NF-κB-SEAP(secreted alkaline phosphatase) reporter (B) and pcDNA3-renilla luciferase reporter (for normalization). After 24 h, cells were treated with TNFα 10 ng/ml for 24 h and dual luciferase or SEAP activities were measured. Representative data in quadruplicate are expressed as relative change compared with empty vector control.

FIG. 16 shows NIBP120 inhibited cell proliferation. MDA-MB-231 cells at 60% confluence were transfected with an empty vector (left panel) or NIBP120 vector (right panel). After 5 days, phase contrast micrographs were taken with a 10× objective.

FIG. 17 shows NIBP mutE and mutG significantly increased cell death in HCT116 cell line. Cells at 80% confluence were transfected with indicated vector. After 5 days, CytoTox-Glo™ Cytotoxicity Assay (promega) was performed. Data represent the percentage of dead cells over total cells.

FIG. 18 shows NIBP mutE and mutG significantly inhibit cell proliferation in HCT116 cell line. Cells at 80% confluence in 6-well plate were transfected with indicated vectors. After 1-6 days, cellTiter-Glo® Luminescent cell Viability Assay (promega) was performed. Data represent relative fold changes compared to pRK empty vector on day 1 after transfection.

FIG. 19 shows Morpholinos against the splicing (spMO) or ATG site (MOatg) led to brain defects (arrows) in zebrafish (top panels), which were rescued by mouse NIBP mRNA (bottom panels).

FIGS. 20(A)-(C) show that NIBP is required for neuronal differentiation of neural stem cells. FIG. 20(A) shows lentivirus-mediated siRNA of NIBP inhibits differentiation in mouse adult neural stem cells. Primary NSCs were infected with lentivirus carrying empty vector pLL3.7 or NIBP-siRNA for 4 h and cultured till formation of secondary neurospheres. Dissociated NSCs were plated on matrigel-coated plate and micrographs were taken at 12 h (top) or 7 d (bottom). FIG. 20(B) shows lentivirus (LV) mediated NIBP over-expression in adult NSCs promotes neuronal differentiation but inhibits astroglial differentiation. Immunocytochemistry was performed at 3 d after differentiation. ** P<0.01 indicates significant difference from corresponding LV-EGFP control. FIG. 20(C) shows real-time PCR analysis in neural stem cells showing that NIBP siRNA (left panels) increased Nestin but decreased PGP9.5 expression. In contrast, NIBP overexpression (right panels) decreased Nestin but increased PGP9.5.

FIG. 21 shows that NIBP knockout in neural stem cells inhibited neurite branching. Dissociated cells from NIBP (f/f) mouse were plated in matrigel-coated plate and infected with Adenovirus carrying Ad-EGFP control vector (top panels) or Ad-Cre-EGFP (bottom panels). After 48 h, photographs were taken under green fluorescent (left panels) or phase contrast (right panels) microscropy. Black arrow show dramatic loss of neurites. White arrows show cells with rich neurites.

FIGS. 22(A)-(D) show tamoxifen-induced Cre-mediated deletion of NIBP in neural stem cells. A schematic diagram shows the DNA structure of NIBP (f/f) and NIBP(−/−) (A). Neurospheres from brain (B) and gut (G) of RCE/NIBP floxed mice were treated with 4-hydroxyl tamoxifen (1 μM, 4 h). After 48 h, genomic DNA was extracted for PCR genotyping with primer 1/3 (A and B). The PCR product was sequenced with primer 1, showing expected sequence after deletion of exons 2-5 (C). The underlined nucleotides represent introduced digestion sites (EcoRI and BamHI/SalI). NIBP protein expression was detected by Western blotting with anti-NIBP antibody (D).

### DETAILED DESCRIPTION OF THE INVENTION

Various terms relating to the systems, methods, and other aspects of the present invention are used throughout the specification and claims. Such terms are to be given their ordinary meaning in the art unless otherwise indicated.

As used in this specification and the appended claims, the singular forms “a,” “an,” and “the” include plural referents unless the content clearly dictates otherwise. Thus, for example, reference to “a cell” includes a combination of two or more cells, and the like.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate to perform the disclosed methods.

The terms “protein” and “polypeptide” are used herein interchangeably, and refer to a polymer of amino acid residues with no limitation with respect to the minimum length of the polymer. The definition includes both full-length proteins and fragments thereof, as well as modifications thereof (e.g., glycosylation, ubiquitinylation, phosphorylation, deletions, additions and substitutions).

The terms “fragment” and “isoform” of a protein are used herein interchangeably, and refer to a polypeptide having an amino acid sequence that is the same as a part, but not all, of the amino acid sequence of the protein.

The term “variant” of a protein as used herein refers to a polypeptide having an amino acid sequence that is the same as the amino acid sequence of the protein except having at least one amino acid modified, for example, deleted, inserted, or replaced. The variant may have an amino acid sequence at least about 80%, 85%, 90%, 95%, 97%, 98%, or 99%, preferably at least about 90%, more preferably at least about 95%, identical to the amino acid sequence of the protein.

It has been observed in accordance with the present invention that different isoforms, more particularly truncation mutants, of the NIK and IKK2 Binding Protein (NIBP) can interact with NIK and IKK2, and also can promote or inhibit the constitutive and stimulated activation of NF-κB. NIBP mutants, as novel inhibitors for NF-κB activation, may hold potential applications for treating many chronic diseases, includin g cancer, neurodegenerative diseases, cardiovascular diseases, inflammatory bowel disease, arthritis, and systematic lupus Erythematosus, among others. Further, NIBP is required for neuronal differentiation, neurite branching, cellular trafficking and mucosal secretion. Accordingly, the invention provides these functional NIBP polypeptides, and polynucleotides encoding these polypeptides, and methods of using these polypeptides and/or polynucleotides.

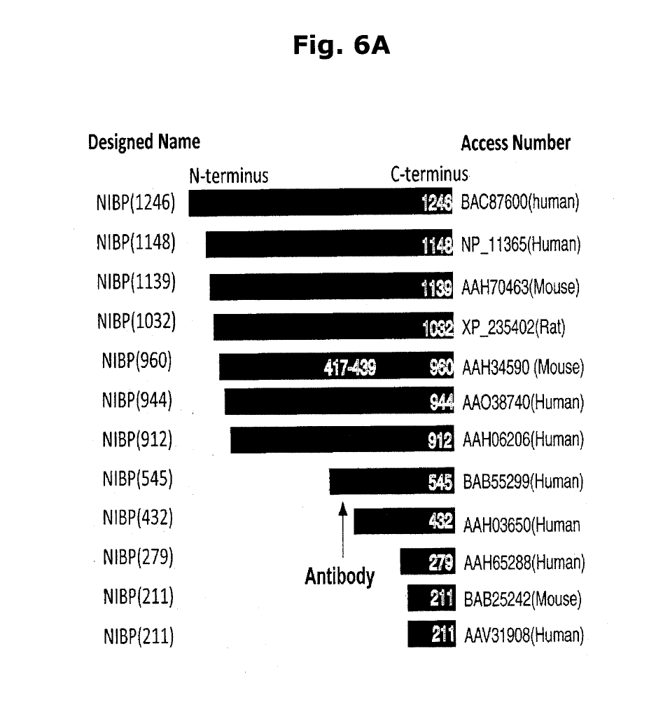

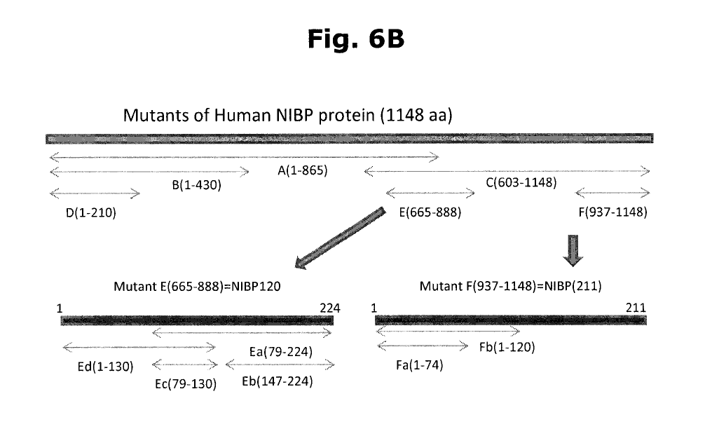

Various NIBP polypeptides, including isoforms are provided. Examples of NIBP polypeptides include NIBP isoforms NIBP1246 (human), NIBP1148 (human), NIBP1139 (mouse), NIBP1032 (rat), NIBP960 (mouse), NIBP944 (human), NIBP912(human), NIBP545 (human), NIBP432 (human), NIBP279 (human), NIBP211 (mouse) and NIBP211 (human) as shown in FIG. 6A, and variants or fragments thereof. Other examples include NIBP polypeptides having an amino acid sequence selected from SEQ ID NOs: 1-7 and 13-16 and variants or fragments thereof. Further examples include NIBP polypeptides comprising residues 1-210, 1-430, 1-865, or 603-1148 of NIBP1148 (SEQ ID NO: 2) and variants or fragments thereof. Variants include addition variants (additional amino acids or amino acid chains added to either or both of the N-terminal or C-terminal end), and variants having a single or multiple amino acid substitutions, deletions, additions, or replacements may retain the biological properties of the base sequence (for example, a marker of one or more stages of breast cancer). The variants may have at least about 80%, 85%, 90%, 95%, 97%, 98%, or 99% identity with a base sequence, for example, a sequence selected from SEQ ID NOs: 1-7 and 13-16. Variants also include fusions with another peptide or polypeptide such as a fusion partner, a protein tag or other chemical moiety. Preferably, the NIBP variants retain the same functions and/or properties as their corresponding base NIBP polypeptides or isoforms.

The NIBP polypeptides may comprise modified amino acids, including those with post-translational modifications, and can comprise chemical modifications, which include but are not limited to covalent attachments of various chemical moieties, sugars, lipids, and the like.

The invention provides fragments of NIBP120 (SEQ ID NO: 3). The fragments can be of any length, and preferably retain the function of inhibiting NF-κB activation. The fragments preferably comprise contiguous amino acids from the base sequence. The fragments can comprise, for example, any number of contiguous amino acids in the range of 10-224 amino acids of SEQ ID NO: 3, or can comprise 30-224 contiguous amino acids of SEQ ID NO: 3, or can comprise 50-224 contiguous amino acids of SEQ ID NO: 3. It is expected that the skilled artisan can review SEQ ID NO: 3, and the gene encoding this polypeptide, SEQ ID NO: 8, and determine what length of fragment to prepare, what contiguous amino acids will make-up the fragment, and what gene will encode the fragment for purposes of recombinant expression. Non-limiting examples of fragments are shown in FIG. 6(B). It is preferred in some aspects that the fragments include at least a portion, preferably all, of the site on SEQ ID NO: 3 that binds to or otherwise interacts with NIK. It is preferred in some aspects that the fragment binds to or otherwise interacts with NIK.

The invention also provides isolated polynucleotides comprising a nucleic acid sequence encoding the NIBP polypeptides. Preferably, the polypeptides comprise a nucleic acid sequence selected from the group consisting of SEQ ID NOs: 8-12, or the complement thereof, and variants thereof. The variants may have at least about 80%, 85%, 90%, 95%, 97%, 98%, or 99% identity with a base sequence, for example, a sequence selected from SEQ ID NOs: 8-12. Preferably, the nucleic acid encodes the amino acid sequence selected from the group consisting of SEQ ID NOs: 1-7 and 13-16. The variants of SEQ ID NOs: 8-12 may comprise degenerate codons in this regard. The polynucleotides can also encode fragments of SEQ ID NO: 1, 2, 3 or 13.

The invention further provides vectors comprising the polynucleotides, nucleic acids, fragments, and variants thereof, including those described and exemplified herein. The vector comprises a nucleic acid sequence encoding a NIBP polypeptide. Preferably, the NIBP polypeptide comprises an amino acid sequence selected from the group consisting of SEQ ID NOs: 1-7 and 13-16. The vectors can be any system suitable for transferring polynucleotides into host cells, and include without limitation, plasmids, cosmids, artificial chromosomes, phagemids, viruses, and the like. Lentivirus vectors are one preferred example of a viral vector. The vectors can be a cloning vector, expression vector, or both.

Host cells comprising the vectors are provided. The host cells can be prokaryotic or eukaryotic cells. Preferably, the host cells are capable of expressing NIBP polypeptides, including those described or exemplified herein. More preferably, the NIBP polypeptides comprise an amino acid sequence selected from the group consisting of SEQ ID NOs: 1-7 and 13-16. In some aspects, the host cell is a cancer cell or cell line, preferably a breast, gut, liver, colorectal, cervix, prostate, lung and brain cancer cell.

One aspect of the invention provides methods for regulating NF-κB activation in a cell. The methods comprise introducing into the cell a vector comprising a nucleic acid sequence encoding a NIBP polypeptide, wherein the NIBP polypeptide is expressed in the cell. The NF-κB activation may be enhanced or inhibited, preferably inhibited. Preferably, the NIBP polypeptide comprises an amino acid sequence selected from the group consisting of SEQ ID NOs: 3-7, preferably SEQ ID NOs: 3 and 5-7, or variants thereof. The NIBP polypeptide is expressed in the cell. For example, the cell can be transformed with a polynucleotide or vector comprising SEQ ID NO: 8, 9, 10, 11, or 12, or variant or fragment thereof, upon which the cell can express the polypeptide encoded by the polynucleotide. The expression in the cell can be constitutive, or can be modulated, for example, by using appropriate promoters or other transcription or translation control mechanisms as known in the art. The cell can be any cell, preferably a cancer cell such as a breast, liver, colorectal, stomach, cervix, prostate, lung or brain cancer cell. The methods can be carried out in vivo or in vitro.

Preferably, the expression of a NIBP polypeptide in the cell or the exposure of the cell to a NIBP polypeptide prevents, slows, or otherwise inhibits the activation of NF-κB in the cell. The affected activation can be constitutive or stimulated/induced. Stimulated/induced activation includes activation of NF-κB through various signal cascades that are activated by chemical, physical, pathophysiological, or other stimulation of the cell. For example, the activation may be stimulated by TNF-α or IL-1β.

The invention also features methods for reversing the cancerous phenotype of a cancer cell. The methods comprise introducing into the cell a vector comprising a nucleic acid sequence encoding a NIBP polypeptide, wherein the NIBP polypeptide is expressed in the cell. Preferably, the NIBP polypeptide comprises an amino acid sequence selected from the group consisting of SEQ ID NOs: 3-7 and 13-16, or variants thereof. Examples of reversing the cancerous phenotype include inhibiting cell proliferation and inducing cell death, for example, programmed (apoptosis) or necrotic cell death.

The invention further features methods for modulating neuronal differentiation of a cell. The methods comprise introducing into the cell a vector comprising a nucleic acid sequence encoding a NIBP polypeptide, wherein the NIBP polypeptide is expressed in the cell. Preferably, the cell is a neural stem cell (NSC), a neural progenitor cell (NPC), or a cell having disrupted NIBP expression. The disruption may be potentially induced by neurodevelopmental disorders, neural injuries and neurodegenerative diseases.

In some embodiments, methods for regulating (e.g., inhibiting) NF-κB activation in a cell, reversing the cancerous phenotype of a cancer cell, or modulating neuronal differentiation of a cell comprise contacting the cell with a NIBP polypeptide. Examples of the NIBP polypeptides include NIBP isoforms NIBP1246 (human), NIBP1148 (human), NIBP1139 (mouse), NIBP1032 (rat), NIBP960 (mouse), NIBP944 (human), NIBP912(human), NIBP545 (human), NIBP432 (human), NIBP279 (human), NIBP211 (mouse) and NIBP211 (human) as shown in FIG. 6A, and variants or fragments thereof. Other examples include NIBP polypeptides having an amino acid sequence selected from SEQ ID NOs: 1-7 and 13-16, and variants or fragments thereof. Further examples include NIBP polypeptides comprising residues 1-210, 1-430, 1-865, or 603-1148 of NIBP1148 (SEQ ID NO: 2) and variants or fragments thereof. The NIBP polypeptide may be synthesized or recombinantly expressed, isolated and/or purified as appropriate, and delivered to the cell. Delivery can include, for example, adding the NIBP polypeptide to the cell culture medium, directly contacting the NIBP polypeptide with specific cell surface receptors, injecting into the cell, or by means of any delivery tag, carrier, vehicle, or technique known and suitable in the art, including a liposome, micelle, fusion tag, antibody, carrier protein, chemical moiety, electroporation, microinjection, viral protein fusions (e.g., TAT, VP22), nanoparticles, and commercial protein delivery reagents such as Chariot™ (Active Motif), BioTrek™ (Stratagene), Transport™ (Cambrex), Provectin™ (Imgenex), BioPorter (Genlantis), and the like. The NIBP polypeptide can be internalized into the cell, preferably into the cytoplasm, by any passive, facilitated, or active processes.

The following examples are provided to describe exemplary aspects of the invention in greater detail. They are intended to illustrate, not to limit, the invention.

### Example 1

### Characterization of NIBP Function

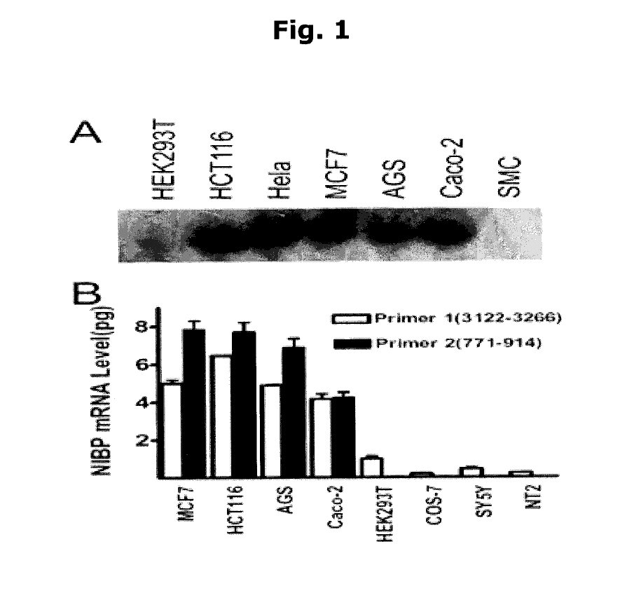

NIBP is highly expressed in cancer cell lines. Northern blot analysis with a probe targeting 1640-2423 bp of the longest cDNA clone identified a single transcript (˜4.5 kb) highly expressed in selected cancer cell lines (FIG. 1A). Absolute quantitative assay by real-time RT-PCR demonstrated high expression of NIBP mRNA in the breast (MCF7) and gut cancer cell lines (HCT116, AGS, Caco-2) (FIG. 1B). The second pair of primers with a PCR product matching 771-914 bp of NIBP(1246) detected mRNA expression only in the cancer cells, suggesting an important role of NIBP N-terminal region in cancer development.

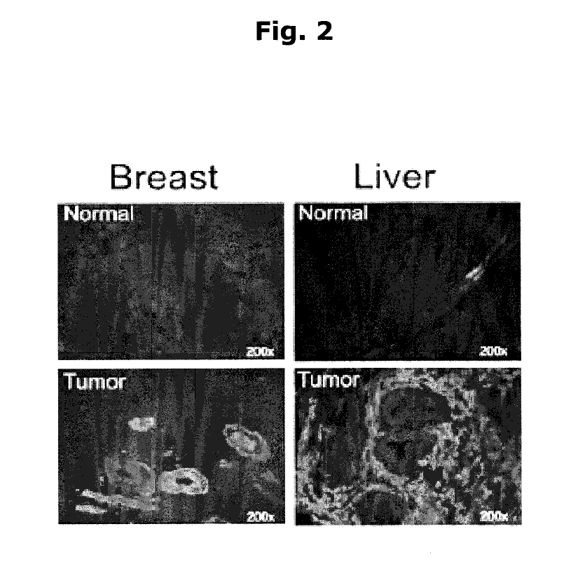

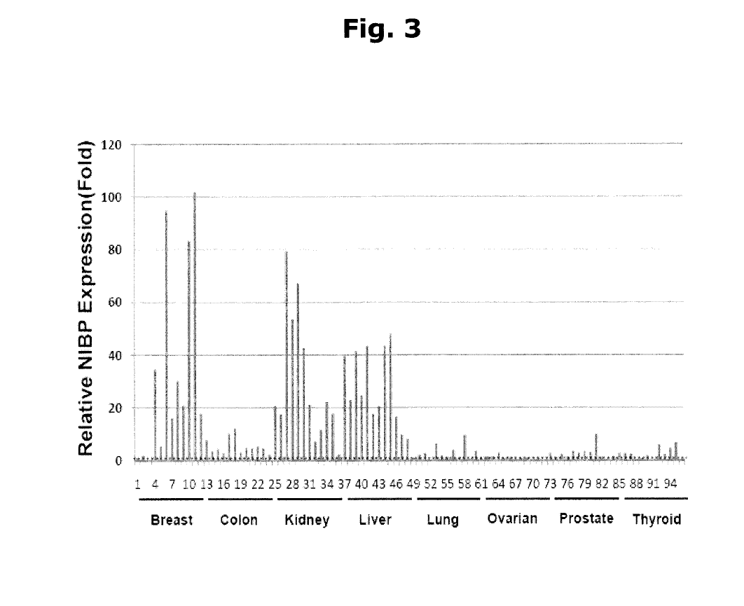

NIBP is highly expressed in human tumor tissues. Unigene analysis suggests that NIBP is widely expressed in various human tumors, with the highest TPM (transcripts per million) in leukemia, breast cancer and gut tumors. Immunohistochemistry staining of human tissue microarray (TMA) showed intensive and extensive NIBP-like immunoreactivity in tumor tissues from breast, liver and other organs (FIG. 2). Human cancer survey tissue-scan real time PCR analysis (CSRT501, Origene) demonstrated the highest increase of NIBP mRNA expression in breast cancer (15-100 fold, FIG. 3).

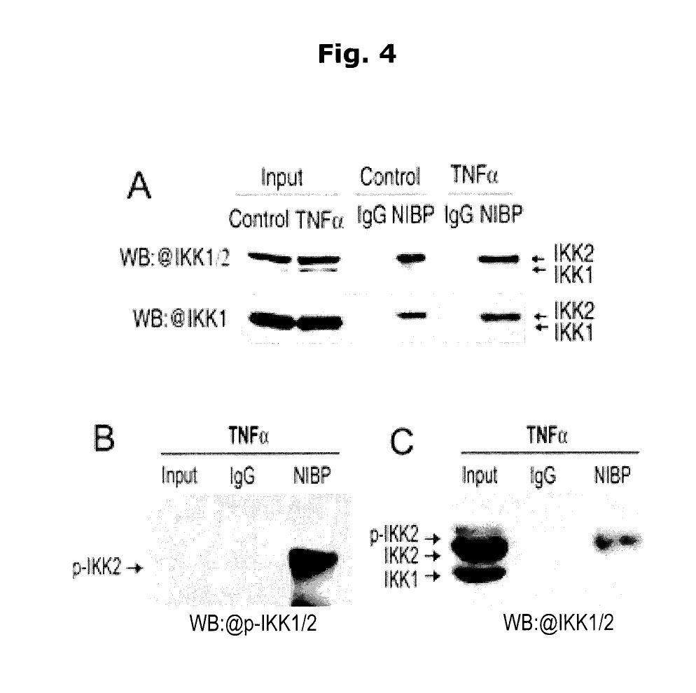

Endogenous NIBP interacts with endogenous IKK2 in breast cancer cells. NIBP association with IKK2, but not IKK1, occurred in MCF7 cells. The level of IKK1 was confirmed by immunoblotting of the same blot (without stripping) with a specific anti-IKK1 antibody. The IKK2 co-immunoprecipitated with NIBP was phosphorylated as shown by the band shift compared to the input and the immunoblotting with anti-phospho IKK1/2(Ser-177/181) antibody (FIG. 4).

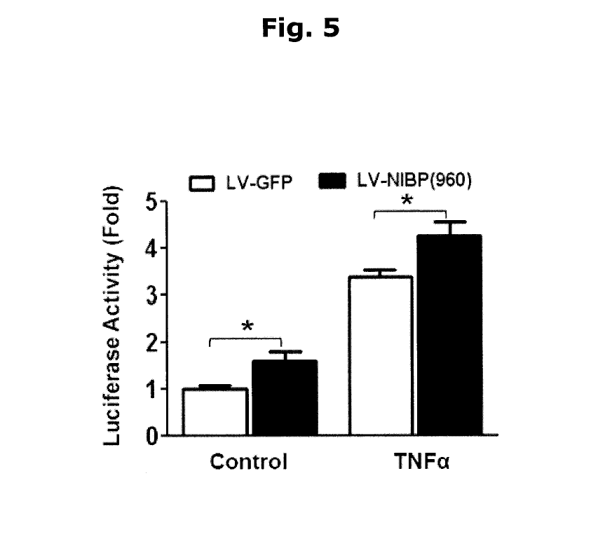

NIBP is required for constitutive and inducible activation of NF-κB in breast cancer cell line. Mouse NIBP(960) enhanced cytokine-induced NF-κB activation in HEK293T cells and colonic cancer cell line. This was corroborated by lentivirus-mediated overexpression of NIBP(960) in MDA-MB-231 cells (FIG. 5), although the increase in NF-κB activation was much weaker than that in HEK293T cells.

NIBP enhances NFκB signaling. Over-expression of NIBP enhanced, whereas knockdown of NIBP inhibited, NF-κB activation induced by TNFα and IL-1β in HEK293T cells. Similar data have been corroborated in cancer cell line (MCF7, MB231, HCT116, AGS, Hela, etc.), neuronal cell line (PC12, enteric neuron) and primary neural stem cells.

NIBP regulates cell proliferation, colony formation and drug-resistance in human cancer cells. To investigate the role of NIBP in regulating colony formation and chemoresistance in gastrointestinal cancer cells, gain or loss-of-function studies were performed in human gastrointestinal cancer cell lines. In HCT116 and AGS cells, stable knockdown of NIBP by lentivirus-mediated siRNA reduced the secretion of IL-8 induced by TNF-α and IL-1β. NIBP knockdown inhibited cell proliferation and colony formation. Camptothecin and Doxorubicin significantly killed AGS cells, which was aggravated by NIBP knockdown. Taxol and Fluorouracil did not kill the AGS cells expressing NIBP ineffective siRNA, but significantly killed the AGS cells stably expressing NIBP effective siRNA. Therefore, NIBP retains the constitutive and inducible activation of NF-κB. Stable knockdown of NIBP effectively inhibits NF-κB activation, cell proliferation and colony formation, as well as sensitizes the cells to chemotherapeutic treatments.

### Example 2

### Isoforms and Mutants of NIBP

The published NIBP has 960 amino acid residues encoded from mouse NIBP isoform I, designated NIBP(960) according to the number of amino acids. Various isoforms or mutants of human NIBP were prepared and expressed in mammalian expression vectors as provided in more detail in the Examples that follow (FIG. 6).

### Example 3

### Interaction Domains between NIBP and IKK2/NIK

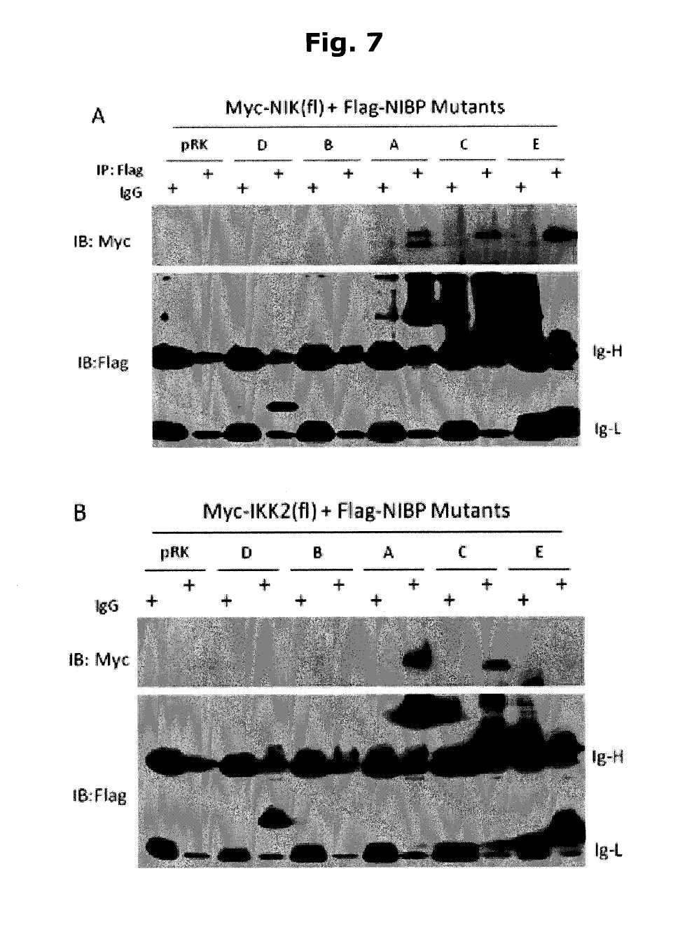

It was previously demonstrated that both NIBP(960) and NIBP(211) interact with IKK2 and NIK. In this Example, the structural-functional relationship between various regions of NIBP and NIK/IKK2 was characterized. As shown in FIG. 7, both A(1-865) and C(603-1148) mutants interacted with NIK and IKK2, whereas B(1-430) and D(1-210) did not interact with either NIK or IKK2, suggesting that the overlapped sequence (603-888) between mutant A and C is responsible for the interaction between NIBP and NIK/IKK2. This region matches the majority of the conserved domain TRS 120 within NIBP, implying that TRS 120 domain (665-888) may interact with NIK/IKK2. Thus, the TRS120 domain was cloned into the pRK-Flag vector, and designated NIBP120 or NIBP-mutE (FIG. 6B).

The mutE(665-888) has strong interaction with NIK (FIG. 7A) but not with IKK2 (FIG. 7B). This suggests that sequence (603-665) within NIBP contains the IKK2-binding site. Therefore, two regions (603-665 and 937-1148) within NIBP(1148) interact with IKK2.

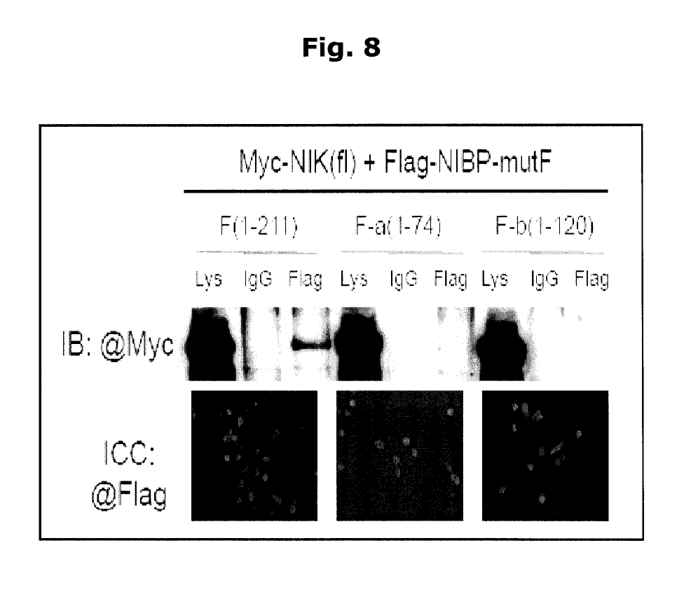

Further deletion studies on NIBP-mutF (equal to NIBP(211)) showed that both sub-mutant Fa(1-74) and Fb(1-120) of NIBP-mutF did not interact with NIK (FIG. 8), implying that NIK-binding site is present in the sub-domain (121-211) of NIBP-mutF. This is consistent with the result from yeast two-hybrid screening showing that NIBP-mutF (133-211) interacts with NIK.

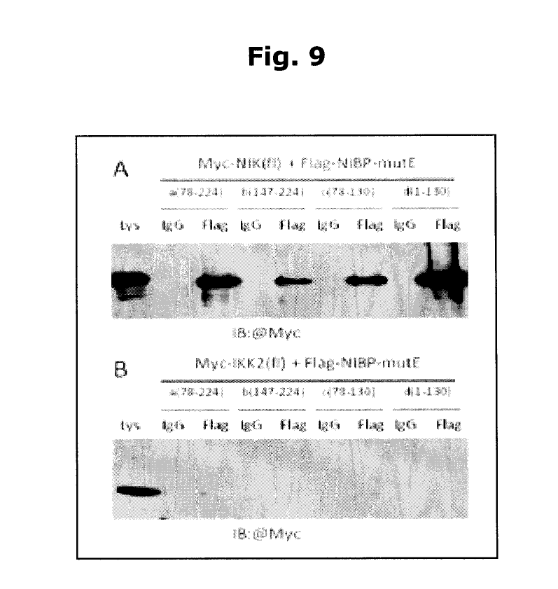

To further analyze the domains within NIBP-mutE(665-888) responsible for NIK binding, four sub-domain mutants were generated by PCR cloning (FIG. 9). These four sub-mutants had no interaction with IKK2, confirming the data as above.

However, they all interacted with NIK to various extents. MutE-a(79-224) and MutE-d(1-130) showed strong interaction with NIK, indicating the region 79-130 (MutE-c) is responsible for NIK binding, though the interaction is weaker than N-terminal region 1-130 (MutE-d). Taken together, the data show that at least three regions (Mut-F, Mut-Ec, and Mut-Eb) within NIBP are capable of interacting with full-length NIK.

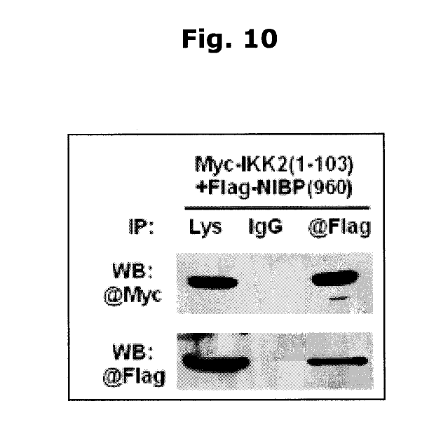

Yeast two-hybrid studies demonstrate that the N terminal region (1-145 aa) of NIK is the binding site for NIBP. To screen which region of IKK2 interacting with NIBP, various deletion mutants of Myc-IKK2 and IKK2-Flag were made and evaluated. The preliminary studies identified N-terminal region (1-103aa) of IKK2 interacting with NIBP (FIG. 10). These data are important for developing novel pharmaceutical targets.

### Example 4

### Function of Selective NIBP Mutants

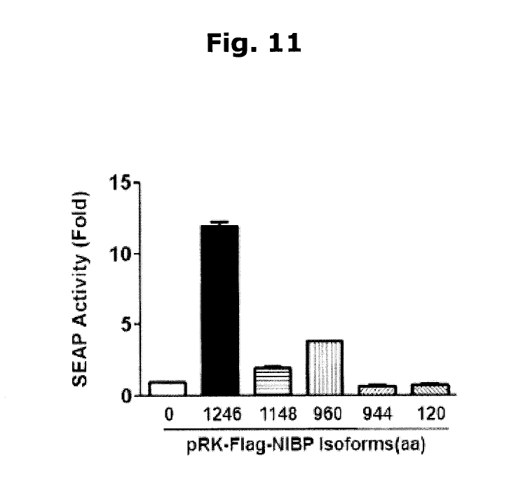

Since NIBP is a novel regulator of NF-κB signaling, the effects of various NIBP isoforms and mutants on cytokine-induced NF-κB activation were examined. As shown in FIG. 5, enhancing effect of NIBP(960) on the constitutive and TNFα-induced NF-κB activation was corroborated in breast cancer cell line MDA-MB-231. In addition, a similar enhancing effect of new isoforms of NIBP(1246) and NIBP(1148) was identified as shown in FIG. 11. Most interestingly, it was discovered that the mutant E (NIBP120) inhibited NF-κB activation in breast cancer cells MB231. A similar effect of NIBP120 was validated in MCF7 and Hela cells.

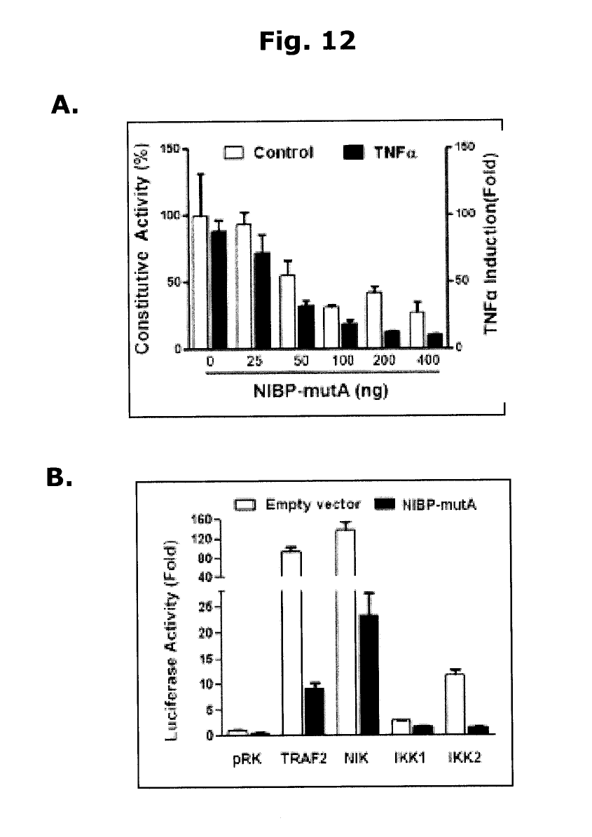

To validate the effect of NIBP120 on NF-κB activation, the dose-response effect in HEK293T cells was evaluated. As shown in FIG. 12, expression of NIBP120 significantly inhibited constitutive and TNFα-induced NF-κB activation. NIBP120 also blocked NF-κB activation induced by over-expression of IKK2 and its upstream signaling components (FIG. 12).

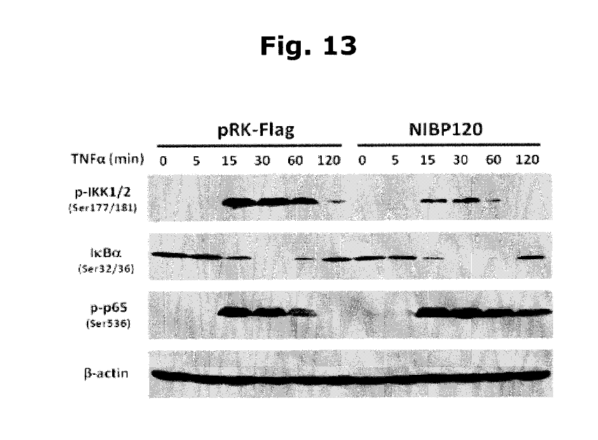

TNFα-induced NF-κB activation is well known to be mediated through the classical IKK2-IκBa/p65 pathway. NIBP120 inhibited TNFα-induced phosphorylation of IKK1/2 (FIG. 13). Generally, IKK2 is phosphorylated by its upstream kinase NIK. Since NIBP120 strongly interacts with NIK but not IKK2, it is believed that NIBP120 may compete with endogenous NIBP (interacting with both NIK and IKK2) by binding to NIK and thus inhibits the activation of IKK2. Surprisingly, NIBP 120 increased TNFα-induced phosphorylation of p65 at Ser-536 (FIG. 13). Although the mechanisms and significance remain unknown, it may reflect the fact that p65 phosphorylation is activated by not only IKK2 but also several other kinases such as IKK1 and RSK1.

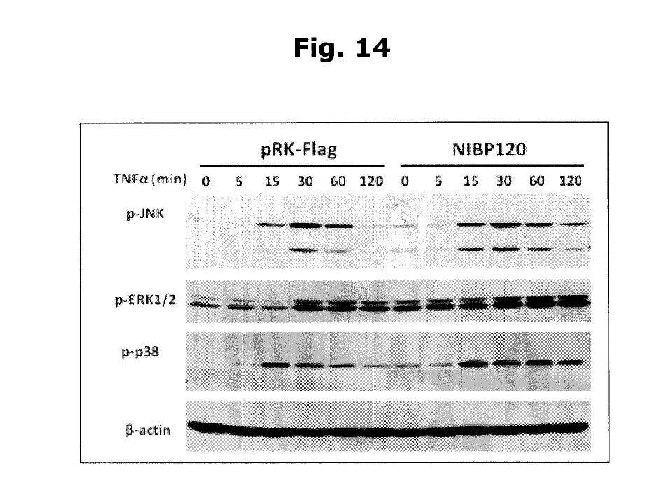

Another interesting finding was that NIBP120 increased the constitutive and TNFα-induced activation of MAPK signaling pathways as determined by the increased phosphorylation in JNK, p38 and ERK1/2 (FIG. 14). This suggests that NIBP120 may have wider functions and applications in addition to NF-κB signaling.

### Example 5

### Functional Analysis of NIBP120 Submutants

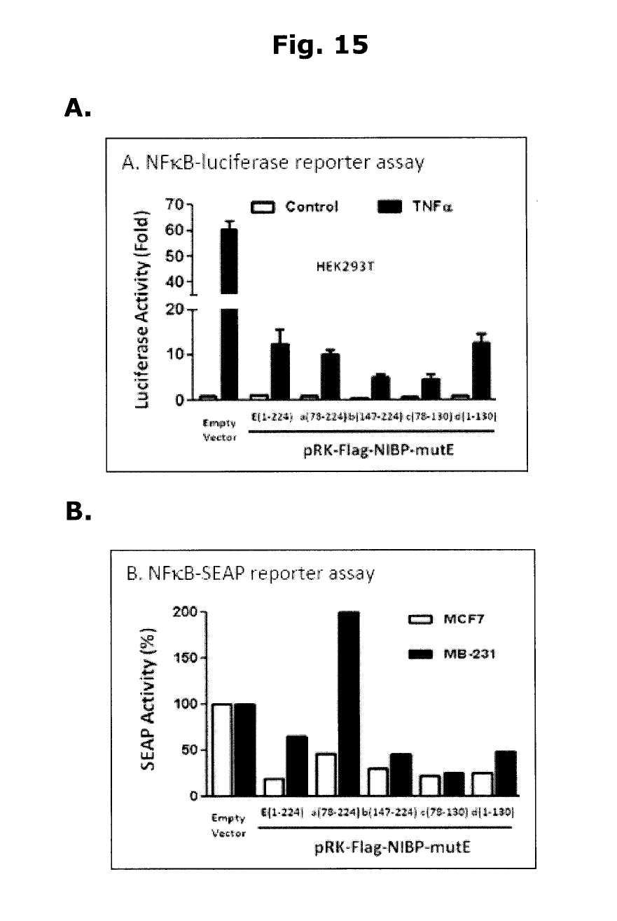

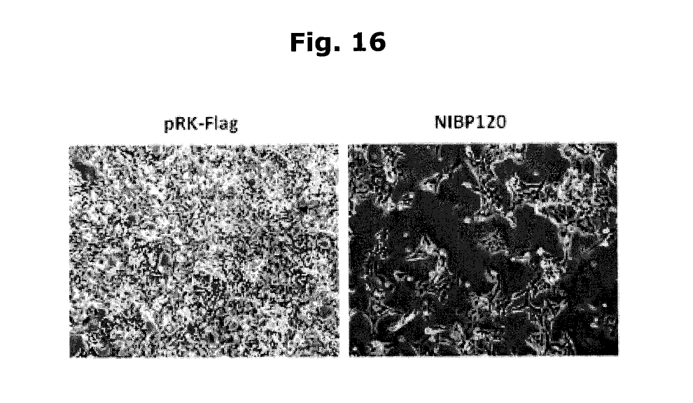

To identify the subdomains of NIBP120 responsible for the inhibitory function, deletion mutants as shown in FIG. 6 were prepared and tested for their effect on NF-κB activation in HEK293T cells and MCF7 and MB-231 cancer cells. As shown in FIG. 15A, NIBP-mutE significantly blocked TNFα-induced NF-κB activation in HEK293T cells, while all four submutants retained the inhibitory effect with further inhibition by the mutEb and mutEc, implying that potential motif within mutEb and mutEc are present for the development of pharmaceutical inhibitors. In the breast cancer cell line, the constitutive activity of NF-κB reporter was significantly inhibited by all submutants in MCF7 and by mutEb, Ec and Ed (FIG. 15B) with the strongest inhibition by mutEc in both cell lines. Therefore, further identification of the motifs within mutEc(79-130) will be greatly valuable.

### Example 6

### Anti-Cancer Effect of NIBP120

As NIBP120 dramatically inhibited NF-κB activation in breast cancer cell line, the effects of NIBP 120 on cell proliferation and colony formation of cancer cells were studied.

NIBP120 was over-expressed in breast cancer cell line MB-231, and cell proliferation and colony formation were then examined. As shown in FIG. 16, NIBP 120 over-expression inhibited cell proliferation and promoted cell death in MB-231 cells.

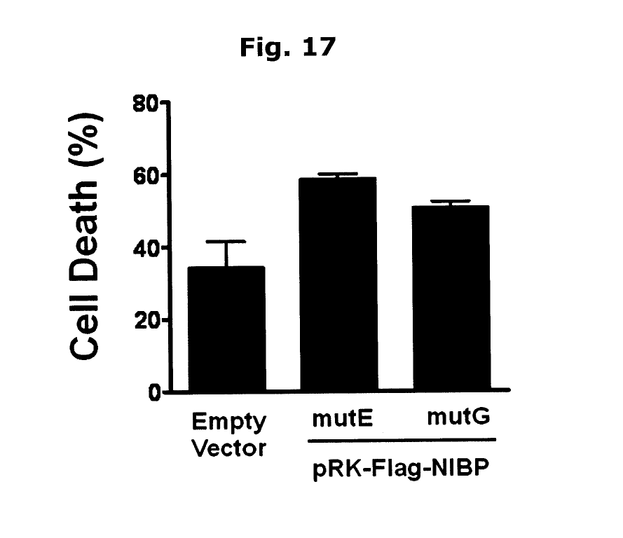

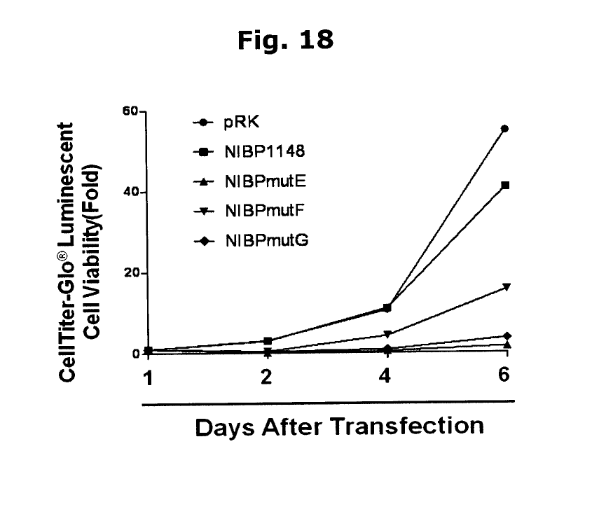

NIBP120 was also over-expressed in colorectal cancer cell line HCT116, and overexpression of NIBP120 (mutE) significantly induced cell death (FIG. 17) and inhibited cell proliferation (FIG. 18). Similar results were observed in NIBPmutE submutants and NIBPmutF submutants. Further, a peptide (65 amino acids) designated NIBPmutG (matching 604-668 residues of NIBP1148) (SEQ ID NO: 16) significantly inhibited the proliferation of cancer cells (FIG. 18).

### Example 7

### Neuronal Expression of NIBP in Adult and Embryonic Brain

NIBP was identified from adult brain cDNA library (Hu et al., 2005). Bioinformatics suggest that NIBP is widely expressed in the nervous system. This is supported by the new in situ hybridization mapping of mouse adult brain showing extensive expression with highest in hippocampus, hypothalamus, cortex. NIBP protein is also extensively expressed in brain (Hu et al., 2005; Mochida et al., 2009).

Immunohistochemic mapping of adult mouse brain showed that NIBP-like immunoreactivity was present in scattered neurons of adult mouse brain (DAB staining) and NIBP was predominantly present in memory-related regions. During mouse embryonic brain development, NIBP-like immunoreactivity was present in migrating neurons during embryonic neurogeneiss starting at E10 and peaking at E12 and transiting to cortical plate (CP) at E13-16, and NIBP expression was undetectable in neuroepithelial cells (E9). These expression profiles suggest that NIBP may promote embryonic neurogenesis and neuronal maturation (Mochida et al., 2009).

### Example 8

### Knocking Down of NIBP Induces Brain Developmental Defects in Zebrafish

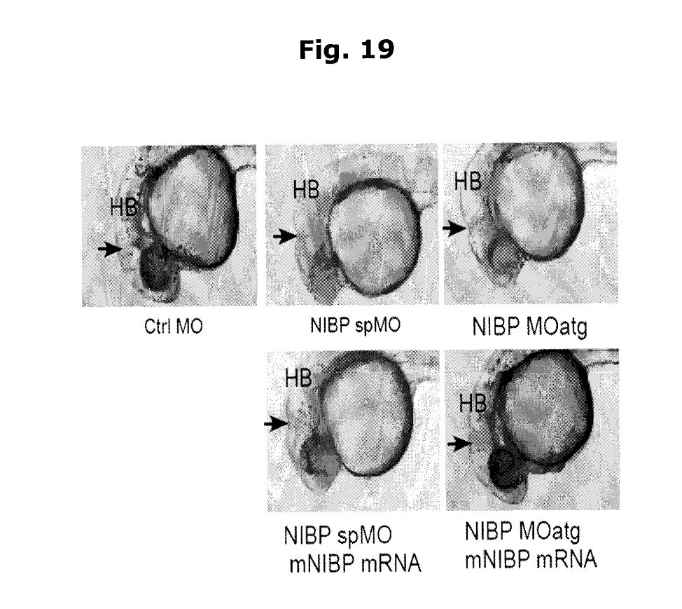

An antisense morpholino (MO) against the splicing of zebrafish NIBP transcript (NIBP spMO) was synthesized. Injection of NIBP spMO did result in abnormal splicing of NIBP RNA, induced an alternative splicing to produce a short alternative mRNA, and dramatically decreased the level of NIBP mRNA. The data demonstrate that the NIBP spMO efficiently block the normal maturation of NIBP mRNA and the subsequent NIBP protein function. Following injection of NIBP spMO, embryos displayed obvious abnormity in embryonic patterning including midbrain and hindbrain tissues. In the severe morphant embryos injected with high concentration of NIBP spMO (8 ng), midbrain, midbrain-hindbrain boundary (MHB), and anterior hindbrain were defected. Less severe ones injected with lower amount of NIBP spMO (2 ng) showed neural tissue disorganization and partial loss (NIBP spMO in FIG. 19). The defects were rescued by co-injection of in vitro synthesized mouse NIBP mRNA (NIBP spMO mNIBP mRNA in FIG. 19), suggesting the specificity of NIBP spMO and the conserved function of NIBP across species. To verify the results, we also injected another morpholino targeting the translation start site of NIBP mRNA (NIBP MOatg) into zebrafish embryos and identified similar phenotypes (NIBP MOatg and NIBP MOatg mNIBP mRNA in FIG. 19).

### Example 9

### NIBP is Required for Neuronal Differentiation of Neural Stem Cells

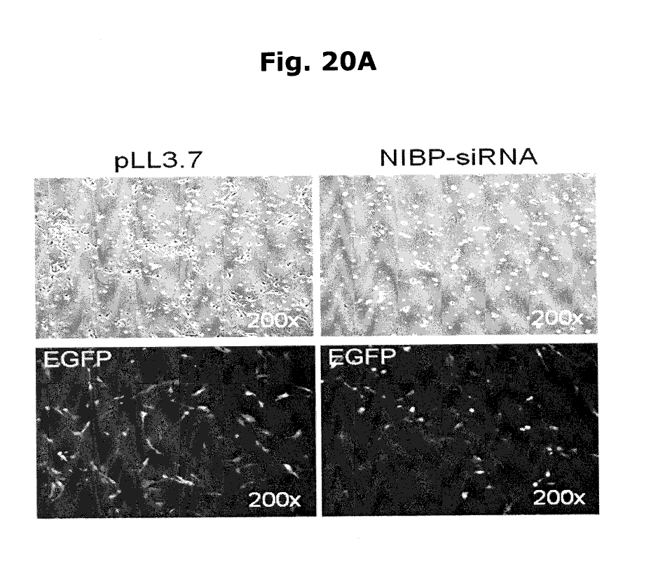

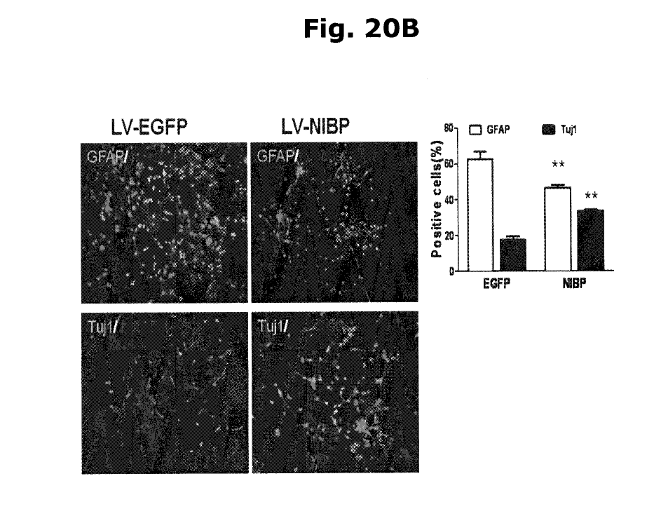

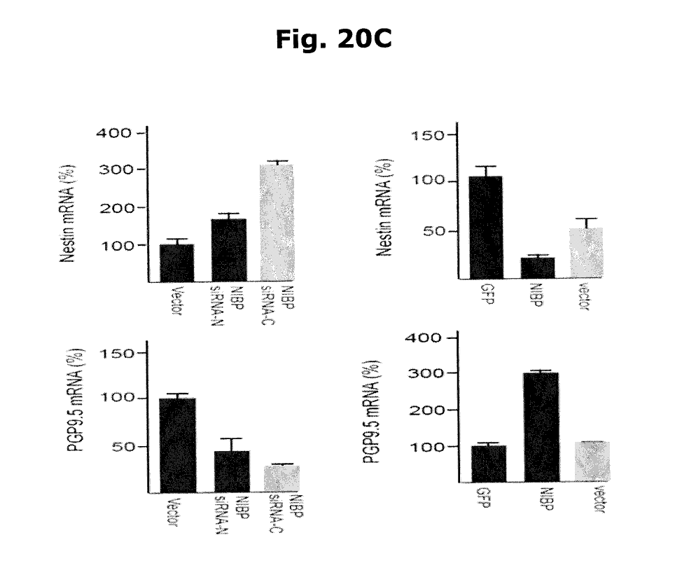

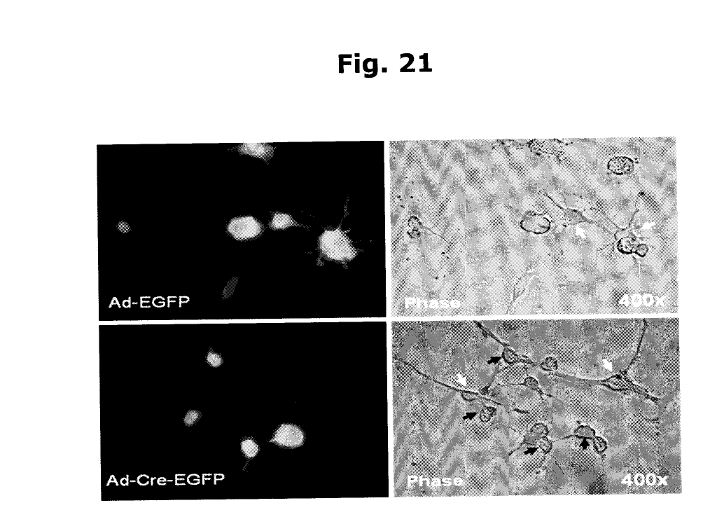

In cultured mouse brain neural stem cells (NSC), lentivirus-mediated stable NIBP knockdown inhibited neuronal differentiation (FIG. 20A) whereas lentivirus-mediated NIBP overexpression promoted neuronal differentiation and inhibited astrocytic differentiation (FIG. 20B), suggesting that NIBP preferentially guides neuronal lineage differentiation. Real-time PCR analysis in neural stem cells showed that NIBP siRNA increased Nestin but decreased PGP9.5 expression (FIG. 20C, left panels) while NIBP overexpression decreased Nestin but increased PGP9.5 (FIG. 20C, right panels).

### Example 10

### Generation of Floxed NIBP Mice

The generation of floxed NIBP conditional knockout mice was initiated using cre-loxP system. A targeting strategy was selected to remove an 8 kb fragment containing exon 2-5. A total 21 kb of NIBP gene was retrieved from 129 BAC clone into pKO vector with A-Red recombineering system and two loxP sites were inserted. Southern blotting and PCR analysis confirmed homologous recombination for both arms of the targeting cassette. Cre-mediated deletion of the floxed fragment in positive embryonic stem (ES) cells was verified by Adeno-Cre-EGFP transduction and PCR genotyping. Two positive clones were selected for microinjection into C57BL/B6 blastocytes. Resulting chimeras crossed with B6 yielded heterozygous floxed NIBP(+/f) mice. Intercrossing of NIBP(+/f) mice generated homozygous floxed NIBP(f/f) mice with the expected Mendelian ratio. The heterozygous and homozygous floxed NIBP mice of both genders were healthy and fertile.

To determine the efficiency of cre-mediated NIBP knockout, NSCs from adult homozygous NIBP(f/f) or wild-type (WT) littermates were cultured and transduced with an Adeno-EGFP vector (FIG. 21, top panels) or an Adeno-Cre-EGFP vector (FIG. 21, top panels). The successful deletion of the designated fragment of NIBP gene by Cre recombination was confirmed by PCR genotyping. Loss of NIBP expression was verified by RT-qPCR and immunocytochemistry. The functional outcome of NIBP knockout in NSCs was reflected by the apparent inhibition of neuronal differentiation and neurite branching detected by Tuj1 immunostaining (FIG. 21, left panels) and phase contrast microscopy (FIG. 21, right panels).

### Example 11

### NIBP Knockout in Cultured NSC/NPCs Led to Blockage of Neuronal Differentiation

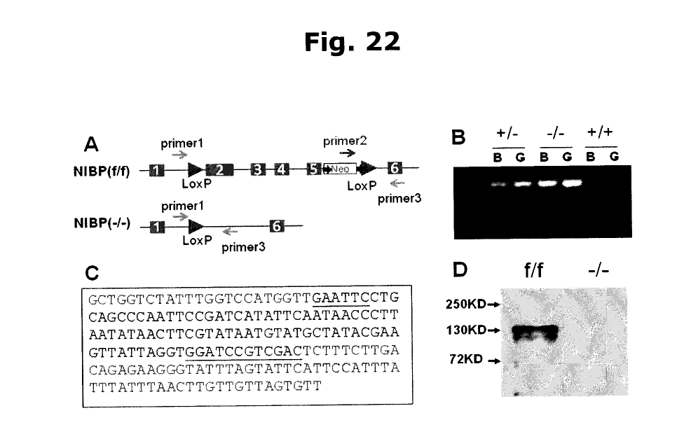

Crossbreeding of homozygous Rosa-Cre-ER(RCE) mice from Jax with NIBP(f/f) mice generated heterozygous double-transgenic RCE(+/−);NIBP(+/f) mice. Intercrossing of F1 offspring yielded homozygous RCE;NIBP(f/f) mice. For preliminary study, SVZ NSCs were cultured from homozygote (f/f), heterozygote(+/f) and wild-type (+/+) of RCE/NIBP littermates. Treatment with 4-hydroxyl-tamoxifen (4-HT) in NSCs efficiently deleted the designated fragment of NIBP gene (FIG. 22B). The resulting PCR product was validated by sequencing (FIG. 22C). By 4 days after induction, NIBP protein was undetectable (FIG. 22D). Neuronal differentiation and neurite branching were dramatically inhibited by NIBP knockout, consistent with Adeno-cre infection (FIG. 21). These preliminary data demonstrate that NIBP is essential for neuronal lineage differentiation and neurite branching.

The present invention is not limited to the embodiments described and exemplified above, but is capable of variation and modification within the scope and range of equivalents of the appended claims.

### REFERENCES

Ahmed, K. M., Dong, S., Fan, M., and Li, J. J. (2006). Nuclear factor-kappaB p65 inhibits mitogen-activated protein kinase signaling pathway in radioresistant breast cancer cells. Mol Cancer Res 4, 945-955.
Bhat-Nakshatri, P., Sweeney, C. J., and Nakshatri, H. (2002). Identification of signal transduction pathways involved in constitutive NF-kappaB activation in breast cancer cells. Oncogene 21, 2066-2078.
Biswas, D. K., Shi, Q., Baily, S., Strickland, I., Ghosh, S., Pardee, A. B., and Iglehart, J. D. (2004). NF-kappa B activation in human breast cancer specimens and its role in cell proliferation and apoptosis. Proc Natl Acad Sci USA 101, 10137-10142.
Bonizzi, G., and Karin, M. (2004). The two NF-kappaB activation pathways and their role in innate and adaptive immunity. Trends Immunol 25, 280-288.
Boyce, B. F., Yao, Z., and Xing, L. (2010). Functions of nuclear factor kappaB in bone. Ann N Y Acad Sci 1192, 367-375.
Buchholz, T. A., Garg, A. K., Chakravarti, N., Aggarwal, B. B., Esteva, F. J., Kuerer, H. M., Singletary, S. E., Hortobagyi, G. N., Pusztai, L., Cristofanilli, M., et al. (2005). The nuclear transcription factor kappaB/bcl-2 pathway correlates with pathologic complete response to doxorubicin-based neoadjuvant chemotherapy in human breast cancer. Clin Cancer Res 11, 8398-8402.
Cao, Y., and Karin, M. (2003). NF-kappaB in mammary gland development and breast cancer. J Mammary Gland Biol Neoplasia 8, 215-223.
Ciucci, A., Gianferretti, P., Piva, R., Guyot, T., Snape, T. J., Roberts, S. M., and Santoro, M. G. (2006). Induction of apoptosis in estrogen receptor-negative breast cancer cells by natural and synthetic cyclopentenones: role of the IkappaB kinase/nuclear factor-kappaB pathway. Mol Pharmacol 70, 1812-1821.
Cogswell, P. C., Guttridge, D. C., Funkhouser, W. K., and Baldwin, A. S., Jr. (2000). Selective activation of NF-kappa B subunits in human breast cancer: potential roles for NF-kappa B2/p52 and for Bcl-3. Oncogene 19, 1123-1131.
Dejardin, E., Bonizzi, G., Bellahcene, A., Castronovo, V., Merville, M. P., and Bours, V. (1995). Highly-expressed p100/p52 (NFκB2) sequesters other NF-kappa B-related proteins in the cytoplasm of human breast cancer cells. Oncogene 11, 1835-1841.
Eddy, S. F., Guo, S., Demicco, E. G., Romieu-Mourez, R., Landesman-Bollag, E., Seldin, D. C., and Sonenshein, G. E. (2005). Inducible IkappaB kinase/IkappaB kinase epsilon expression is induced by CK2 and promotes aberrant nuclear factor-kappaB activation in breast cancer cells. Cancer Res 65, 11375-11383.
Frelin, C., Imbert, V., Griessinger, E., Peyron, A. C., Rochet, N., Philip, P., Dageville, C., Sirvent, A., Hummelsberger, M., Berard, E., et al. (2005). Targeting NF-kappaB activation via pharmacologic inhibition of IKK2-induced apoptosis of human acute myeloid leukemia cells. Blood 105, 804-811.
Ghobrial, I. M., Roccaro, A., Hong, F., Weller, E., Rubin, N., Leduc, R., Rourke, M., Chuma, S., Sacco, A., Jia, X., et al. (2010). Clinical and translational studies of a phase II trial of the novel oral Akt inhibitor perifosine in relapsed or relapsed/refractory Waldenstrom's macroglobulinemia. Clin Cancer Res 16, 1033-1041.
Greten, F. R., Eckmann, L., Greten, T. F., Park, J. M., Li, Z. W., Egan, L. J., Kagnoff, M. F., and Karin, M. (2004). IKKbeta links inflammation and tumorigenesis in a mouse model of colitis-associated cancer. Cell 118, 285-296.
Hacker, H., and Karin, M. (2006). Regulation and function of IKK and IKK-related kinases. Sci STKE 2006, re13.
Haffner, M. C., Berlato, C., and Doppler, W. (2006). Exploiting our knowledge of NF-kappaB signaling for the treatment of mammary cancer. J Mammary Gland Biol Neoplasia 11, 63-73.
Hu, M. C., and Hung, M. C. (2005). Role of IkappaB kinase in tumorigenesis. Future Oncol 1, 67-78.
Hu, M. C., Lee, D. F., Xia, W., Golfman, L. S., Ou-Yang, F., Yang, J. Y., Zou, Y., Bao, S., Hanada, N., Saso, H., et al. (2004). IkappaB kinase promotes tumorigenesis through inhibition of forkhead FOX03a. Cell 117, 225-237.
Hu, W. H., Pendergast, J. S., Mo, X. M., Brambilla, R., Bracchi-Ricard, V., Li, F., Walters, W. M., Blits, B., He, L., Schaal, S. M., et al. (2005). NIBP, a novel NIK and IKK(beta)-binding protein that enhances NF-(kappa)B activation. J Biol Chem 280, 29233-29241.
Huber, M. A., Azoitei, N., Baumann, B., Grunert, S., Sommer, A., Pehamberger, H., Kraut, N., Beug, H., and Wirth, T. (2004). NF-kappaB is essential for epithelial-mesenchymal transition and metastasis in a model of breast cancer progression. J Clin Invest 114, 569-581.
Jackson-Bernitsas, D. G., Ichikawa, H., Takada, Y., Myers, J. N., Lin, X. L., Darnay, B. G., Chaturvedi, M. M., and Aggarwal, B. B. (2006). Evidence that TNF-TNFR1-TRADD-TRAF2-RIP-TAK1-IKK pathway mediates constitutive NF-kappaB activation and proliferation in human head and neck squamous cell carcinoma. Oncogene.
Karin, M. (2006). Nuclear factor-kappaB in cancer development and progression. Nature 441, 431-436.
Karin, M., and Greten, F. R. (2005). NF-kappaB: linking inflammation and immunity to cancer development and progression. Nat Rev Immunol 5, 749-759.
Kent, J. W., Jr., Peterson, C. P., Dyer, T. D., Almasy, L., and Blangero, J. (2009). Genome-wide discovery of maternal effect variants. BMC Proc 3 Suppl 7, S19.
Kim, D. W., Sovak, M. A., Zanieski, G., Nonet, G., Romieu-Mourez, R., Lau, A. W., Hafer, L. J., Yaswen, P., Stampfer, M., Rogers, A. E., et al. (2000). Activation of NF-kappaB/Rel occurs early during neoplastic transformation of mammary cells. Carcinogenesis 21, 871-879.
Kim, H. J., Hawke, N., and Baldwin, A. S. (2006). NF-kappaB and IKK as therapeutic targets in cancer. Cell Death Differ 13, 738-747.
Kim, J. C., Kim, S. Y., Roh, S. A., Cho, D. H., Kim, D. D., Kim, J. H., and Kim, Y. S. (2008). Gene expression profiling: canonical molecular changes and clinicopathological features in sporadic colorectal cancers. World J Gastroenterol 14, 6662-6672.
Koifman, A., Feigenbaum, A., Bi, W., Shaffer, L. G., Rosenfeld, J., Blaser, S., and Chitayat, D. (2010). A homozygous deletion of 8q24.3 including the NIBP gene associated with severe developmental delay, dysgenesis of the corpus callosum, and dysmorphic facial features. Am J Med Genet A 152A, 1268-1272.
Kummel, D., Oeckinghaus, A., Wang, C., Krappmann, D., and Heinemann, U. (2008). Distinct isocomplexes of the TRAPP trafficking factor coexist inside human cells. FEBS Lett 582, 3729-3733.
Lin, Y., Bai, L., Chen, W., and Xu, S. (2010). The NF-kappaB activation pathways, emerging molecular targets for cancer prevention and therapy. Expert Opin Ther Targets 14, 45-55.
Luo, J. L., Kamata, H., and Karin, M. (2005). IKK/NF-kappaB signaling: balancing life and death—a new approach to cancer therapy. J Clin Invest 115, 2625-2632.
Mancino, A., and Lawrence, T. (2010). Nuclear factor-kappaB and tumor-associated macrophages. Clin Cancer Res 16, 784-789.
Mantovani, A., and Balkwill, F. (2006). RaIB signaling: a bridge between inflammation and cancer. Cell 127, 42-44.
Mir, A., Kaufman, L., Noor, A., Motazacker, M. M., Jamil, T., Azam, M., Kahrizi, K., Rafiq, M. A., Weksberg, R., Nasr, T., et al. (2009). Identification of mutations in TRAPPC9, which encodes the NIK- and IKK-beta-binding protein, in nonsyndromic autosomal-recessive mental retardation. Am J Hum Genet 85, 909-915.
Mochida, G. H., Mahajnah, M., Hill, A. D., Basel-Vanagaite, L., Gleason, D., Hill, R. S., Bodell, A., Crosier, M., Straussberg, R., and Walsh, C. A. (2009). A truncating mutation of TRAPPC9 is associated with autosomal-recessive intellectual disability and postnatal microcephaly. Am J Hum Genet 85, 897-902.
Montagut, C., Tusquets, I., Ferrer, B., Corominas, J. M., Bellosillo, B., Campas, C., Suarez, M., Fabregat, X., Campo, E., Gascon, P., et al. (2006). Activation of nuclear factor-kappa B is linked to resistance to neoadjuvant chemotherapy in breast cancer patients. Endocr Relat Cancer 13, 607-616.
Nakshatri, H., Bhat-Nakshatri, P., Martin, D. A., Goulet, R. J., Jr., and Sledge, G. W., Jr. (1997). Constitutive activation of NF-kappaB during progression of breast cancer to hormone-independent growth. Mol Cell Biol 17, 3629-3639.
O'Sullivan, N. C., Croydon, L., McGettigan, P. A., Pickering, M., and Murphy, K. J. (2010). Hippocampal region-specific regulation of NF-kappaB may contribute to learning-associated synaptic reorganisation. Brain Res Bull 81, 385-390.
Pacifico, F., and Leonardi, A. (2006). NF-kappaB in solid tumors. Biochem Pharmacol 72, 1142-1152.
Park, B. K., Zhang, H., Zeng, Q., Dai, J., Keller, E. T., Giordano, T., Gu, K., Shah, V., Pei, L., Zarbo, R. J., et al. (2007). NF-kappaB in breast cancer cells promotes osteolytic bone metastasis by inducing osteoclastogenesis via GM-CSF. Nat Med 13, 62-69.
Patel, N. M., Nozaki, S., Shortie, N. H., Bhat-Nakshatri, P., Newton, T. R., Rice, S., Gelfanov, V., Boswell, S. H., Goulet, R. J., Jr., Sledge, G. W., Jr., et al. (2000). Paclitaxel sensitivity of breast cancer cells with constitutively active NF-kappaB is enhanced by IkappaBalpha super-repressor and parthenolide. Oncogene 19, 4159-4169.
Perkins, N. D. (2007). Integrating cell-signalling pathways with NF-kappaB and IKK function. Nat Rev Mol Cell Biol 8, 49-62.
Philippe, O., Rio, M., Carioux, A., Plaza, J. M., Guigue, P., Molinari, F., Boddaert, N., Bole-Feysot, C., Nitschke, P., Smahi, A., et al. (2009). Combination of linkage mapping and microarray-expression analysis identifies NF-kappaB signaling defect as a cause of autosomal-recessive mental retardation. Am J Hum Genet 85, 903-908.
Ping, B., He, X., Xia, W., Lee, D. F., Wei, Y., Yu, D., Mills, G., Shi, D., and Hung, M. C. (2006). Cytoplasmic expression of p21CIP1/WAF1 is correlated with IKKbeta overexpression in human breast cancers. Int J Oncol 29, 1103-1110.
Riendeau, N. (2009). Autism Spectrum Disorders: Identification of Novel Microdeletions and Microduplications and their Associated Phenotypes. Graduate Thesis, The University Of British Columbia, 56-59.
Romieu-Mourez, R., Landesman-Bollag, E., Seldin, D. C., Traish, A. M., Mercurio, F., and Sonenshein, G. E. (2001). Roles of IKK kinases and protein kinase CK2 in activation of nuclear factor-kappaB in breast cancer. Cancer Res 61, 3810-3818.
Ross, C. W., Ouillette, P. D., Saddler, C. M., Shedden, K. A., and Malek, S. N. (2007). Comprehensive analysis of copy number and allele status identifies multiple chromosome defects underlying follicular lymphoma pathogenesis. Clin Cancer Res 13, 4777-4785.
Ruocco, M. G., and Karin, M. (2005). IKK{beta} as a target for treatment of inflammation induced bone loss. Ann Rheum Dis 64 Suppl 4, iv81-85.
Sovak, M. A., Bellas, R. E., Kim, D. W., Zanieski, G. J., Rogers, A. E., Traish, A. M., and Sonenshein, G. E. (1997). Aberrant nuclear factor-kappaB/Rel expression and the pathogenesis of breast cancer. J Clin Invest 100, 2952-2960.
Tanaka, A., Muto, S., Konno, M., Itai, A., and Matsuda, H. (2006). A new IkappaB kinase beta inhibitor prevents human breast cancer progression through negative regulation of cell cycle transition. Cancer Res 66, 419-426.
Tokarev, A. A., Taussig, D., Sundaram, G., Lipatova, Z., Liang, Y., Mulholland, J. W., and Segev, N. (2009). TRAPP II complex assembly requires Trs33 or Trs65. Traffic 10, 1831-1844.
Wang, L. C., Okitsu, C. Y., and Zandi, E. (2005). Tumor necrosis factor alpha-dependent drug resistance to purine and pyrimidine analogues in human colon tumor cells mediated through IKK. J Biol Chem 280, 7634-7644.
Wong, E. T., and Tergaonkar, V. (2009). Roles of NF-kappaB in health and disease: mechanisms and therapeutic potential. Clin Sci (Lond) 116, 451-465.
Yamaguchi, N., Ito, T., Azuma, S., Ito, E., Honma, R., Yanagisawa, Y., Nishikawa, A., Kawamura, M., Imai, J., Watanabe, S., et al. (2009). Constitutive activation of nuclear factor-kappaB is preferentially involved in the proliferation of basal-like subtype breast cancer cell lines. Cancer Sci 100, 1668-1674.
Yoshida, T., Kato, K., Yokoi, K., Oguri, M., Watanabe, S., Metoki, N., Yoshida, H., Satoh, K., Aoyagi, Y., Nozawa, Y., et al. (2010). Association of genetic variants with hemorrhagic stroke in Japanese individuals. Int J Mol Med 25, 649-656.
Zahoor, M. A., Yamane, D., Mohamed, Y. M., Suda, Y., Kobayashi, K., Kato, K., Tohya, Y., and Akashi, H. (2010). Bovine viral diarrhea virus non-structural protein 5A interacts with NIK- and IKKbeta-binding protein. J Gen Virol 91, 1939-1948.

Sequence Listing

SEQ ID NO: 1 (Human NIBP/1246)

| | |
|---|---|
| 1 | mvpagdqdra phrgkpaqag artsrasral rswrrsqaar atvthprggh drgshggyre |
| 61 | ghrgcrrdpq wasagpppls fteevkfelr alkdwdfkms vpdymqcaed hqtllvvvqp |
| 121 | vgivseenff riykricsvs qisvrdsqrv lyiryrhhyp pennewgdfq thrkvvglit |
| 181 | itdcfsakdw pqtfekfhvq keiygstlyd srlfvfglqg eiveqprtdv afypnyedcq |
| 241 | tvekriedfi eslfivlesk rldratdksg dkipllcvpf ekkdfvgldt dsrhykkrcq |
| 301 | grmrkhvgdl clqagmlqds lvhyhmsvel lrsvndflwl gaaleglcsa sviyhypggt |
| 361 | ggksgarrfq gstlpaeaan rhrpgaqevl idpgalttng inpdtsteig raknclsped |
| 421 | iidkykeais yyskyknagv ieleacikav rvlaiqkrsm easeflqnav yinlrqlsee |
| 481 | ekiqrysils elyeligfhr ksaffkrvaa mqcvapsiae pgwracykll letlpgysls |
| 541 | ldpkdfsrgt hrgwaavqmr llhelvyasr rmgnpalsvr hlsfllqtml dflsdqekkd |
| 601 | vaqslenyts kcpgtmepia lpggltlppv pftklpivrh vkllnlpasl rphkmksllg |
| 661 | qnvstkspfi yspiiahnrg eernkkidfq wvqgdvcevq lmvynpmpfe lrvenmgllt |
| 721 | sgvefeslpa alslpaesgl ypvtlvgvpq ttgtitvngy httvfgvfsd clldnlpgik |
| 781 | tsgstvevip alprlqists lprsahslqp ssgdeistnv svqlyngesq qliiklenig |
| 841 | mepleklevt skvlttkekl ygdflswkle etlaqfplqp gkvatftini kvkldfscqe |
| 901 | nllqdlsddg isvsgfplss pfrqvvrprv egkpvnppes nkagdyshvk tleavlnfky |
| 961 | sggpghtegy yrnlslglhv evepsvfftr vstlpatstr qchllldvfn steheltvst |
| 1021 | rssealilha gecqrmaiqv dkfnfesfpe spgekgqfan pkqleeerre argleihskl |
| 1081 | gicwripslk rsgeasvegl lnqlvlehlq laplqwdvlv dgqpcdreav aacqvgdpvr |
| 1141 | levrltnrsp rsvgpfaltv vpfqdhqngv hnydlhdtvs fvgsstfyld avqpsgqsac |
| 1201 | lgallflytg dfflhirfhe dstskelpps wfclpsvhvc aleaqa |

SEQ ID NO: 2 (Human NIBP/1148)

| | |
|---|---|
| 1 | msvpdymqca edhqtllvvv qpvgivseen ffriykrics vsqisvrdsq rvlyiryrhh |
| 61 | yppennewgd fqthrkvvgl ititdcfsak dwpqtfekfh vqkeiygstl ydsrlfvfgl |
| 121 | qgeiveqprt dvafypnyed cqtvekried fieslfivle skrldratdk sgdkipllcv |
| 181 | pfekkdfvgl dtdsrhykkr cqgrmrkhvg dlclqagmlq dslvhyhmsv ellrsvndfl |
| 241 | wlgaaleglc sasviyhypg gtggksgarr fqgstlpaea anrhrpgaqe vlidpgaltt |
| 301 | nginpdtste igraknclsp ediidkykea isyyskykna gvieleacik avrvlaiqkr |
| 361 | smeaseflqn avyinlrqls eeekiqrysi lselyeligf hrksaffkrv aamqcvapsi |
| 421 | aepgwracyk llletlpgys lsldpkdfsr gthrgwaavq mrllhelvya srrmgnpals |
| 481 | vrhlsfllqt mldflsdqek kdvaqsleny tskcpgtmep ialpggltlp pvpftklpiv |
| 541 | rhvkllnlpa slrphkmksl lgqnvstksp fiyspiiahn rgeernkkid fqwvqgdvce |
| 601 | vqlmvynpmp felrvenmgl ltsgvefesl paalslpaes glypvtlvgv pqttgtitvn |
| 661 | gyhttvfgvf sdclldnlpg iktsgstvev ipalprlqis tslprsahsl qpssgdeist |
| 721 | nvsvqlynge sqqliiklen igmeplekle vtskvlttke klygdflswk leetlaqfpl |
| 781 | qpgkvatfti nikvkldfsc qenllqdlsd dgisvsgfpl sspfrqvvrp rvegkpvnpp |
| 841 | esnkagdysh vktleavlnf kysggpghte gyyrnlslgl hvevepsvff trvstlpats |
| 901 | trqchllldv fnsteheltv strssealil hagecqrmai qvdkfnfesf pespgekgqf |
| 961 | anpkqleeer reargleihs klgicwrips lkrsgeasve gllnqlvleh lqlaplqwdv |
| 1021 | lvdgqpcdre avaacqvgdp vrlevrltnr sprsvgpfal tvvpfqdhqn gvhnydlhdt |
| 1081 | vsfvgsstfy ldavqpsgqs aclgallfly tgdfflhirf hedstskelp pswfclpsvh |
| 1141 | vcaleaqa |

SEQ ID NO: 3 (NIBP120)

| | |
|---|---|
| 1 | tvfgvfsdcl ldnlpgikts gstvevipal prlqistslp rsahslqpss gdeistnvsv |
| 61 | qlyngesqql iiklenigme pleklevtsk vlttkeklyg dflswkleet laqfplqpgk |
| 121 | vatftinikv kldfscqenl lqdlsddgis vsgfplsspf rqvvrprveg kpvnppesnk |
| 181 | agdyshvktl eavlnfkysg gpghtegyyr nlslglhvev epsv |

SEQ ID NO: 4 (Residues 79-224 of NIBP120)

| | |
|---|---|
| 1 | mepleklevt skvlttkekl ygdflswkle etlaqfplqp gkvatftini kvkldfscqe |
| 61 | nllqdlsddg isvsgfplss pfrqvvrprv egkpvnppes nkagdyshvk tleavlnfky |
| 121 | sggpghtegy yrnlslglhv evepsv |

SEQ ID NO: 5 (Residues 1-130 of NIBP120)

| | |
|---|---|
| 1 | tvfgvfsdcl ldnlpgikts gstvevipal prlqistslp rsahslqpss gdeistnvsv |
| 61 | qlyngesqql iiklenigme pleklevtsk vlttkeklyg dflswkleet laqfplqpgk |
| 121 | vatftinikv |

SEQ ID NO: 6 (Residues 79-130 of NIBP120)

| | |
|---|---|
| 1 | mepleklevt skvlttkekl ygdflswkle etlaqfplqp gkvatftini kv |

SEQ ID NO: 7 (Residues 147-224 of NIBP120)

| | |
|---|---|
| 1 | dgisysgfpl sspfrqvvrp rvegkpvnpp esnkagdysh vktleavlnf kysggpghte |
| 61 | gyyrnlslgl hvevepsv |

SEQ ID NO: 8 (Nucleic Acid Sequence of NIBP120)

| | |
|---|---|
| 1 | acggtcttcg gtgtgttcag tgactgtttg ctggataacc tgccgggaat aaaaaccagt |
| 61 | ggctccacag tggaagtcat tcccgcgttg ccaagactgc agatcagcac ctctctgccc |
| 121 | agatctgcac attcattgca accttcttct ggtgatgaaa tatctactaa tgtatctgtc |
| 181 | cagctttaca atggagaaag tcagcaacta atcattaaat tggaaaatat tggaatggaa |
| 241 | ccattggaga aactggaggt cacctcgaaa gttctcacca ctaaagaaaa attgtatggc |
| 301 | gacttcttga gctggaagct agaggaaacc cttgcccagt tccctttgca gcctgggaag |
| 361 | gtggccacgt tcacaatcaa catcaaagtg aagctggatt tctcctgcca ggagaatctc |
| 421 | ctgcaggatc tcagtgatga tggaatcagt gtgagtggct ttcccctgtc cagtcctttt |
| 461 | cggcaggtcg ttcggccccg agtggagggc aaacctgtga acccacccga gagcaacaaa |
| 541 | gcaggcgact acagccacgt gaagaccctg gaagctgtcc tgaatttcaa atactctgga |
| 601 | ggcccgggcc acactgaagg atattacagg aatctctccc tggggctgca tgtagaagtc |
| 661 | gagccgtctg ta |

SEQ ID NO: 9 (Nucleic Acid Sequence for Residues 79-224 of NIBP120)

| | |
|---|---|
| 1 | atggaaccat tggagaaact ggaggtcacc tcgaaagttc tcaccactaa agaaaaattg |
| 61 | tatggcgact tcttgagctg gaagctagag gaaacccttg cccagttccc tttgcagcct |
| 121 | gggaaggtgg ccacgttcac aatcaacatc aaagtgaagc tggatttctc ctgccaggag |
| 181 | aatctcctgc aggatctcag tgatgatgga atcagtgtga gtggctttcc cctgtccagt |
| 241 | ccttttcggc aggtcgttcg gccccgagtg gagggcaaac ctgtgaaccc acccgagagc |
| 301 | aacaaagcag gcgactacag ccacgtgaag accctggaag ctgtcctgaa tttcaaatac |
| 361 | tctggaggcc cgggccacac tgaaggatat tacaggaatc tctccctggg gctgcatgta |
| 421 | gaagtcgagc cgtctgca |

SEQ ID NO: 10 (Nucleic Acid Sequence for Residues 1-130 of NIBP120)

| | |
|---|---|
| 1 | acggtcttcg gtgtgttcag tgactgtttg ctggataacc tgccgggaat aaaaaccagt |
| 61 | ggctccacag tggaagtcat tcccgcgttg ccaagactgc agatcagcac ctctctgccc |
| 121 | agatctgcac attcattgca accttcttct ggtgatgaaa tatctactaa tgtatctgtc |
| 181 | cagctttaca atggagaaag tcagcaacta atcattaaat tggaaaatat tggaatggaa |
| 241 | ccattggaga aactggaggt cacctcgaaa gttctcacca ctaaagaaaa attgtatggc |
| 301 | gacttcttga gctggaagct agaggaaacc cttgcccagt tccctttgca gcctgggaag |
| 361 | gtggccacgt tcacaatcaa catcaaagtg |

SEQ ID NO: 11 (Nucleic Acid Sequence for Residues 79-130 of NIBP120)

| | |
|---|---|
| 1 | atggagaaag tcagcaacta atcattaaat tggaaaatat tggaatggaa ccattggaga |
| 61 | aactggaggt cacctcgaaa gttctcacca ctaaagaaaa attgtatggc gacttcttga |
| 121 | gctggaagct agaggaaacc cttgcccagt tccctttgca gcctgggaag gtggccacgt |
| 181 | tcacaatcaa catcaaagtg |

SEQ ID NO: 12 (Nucleic Acid Sequence for Residues 147-224 of NIBP120)

| | |
|---|---|
| 1 | gatggaatca gtgtgagtgg ctttcccctg tccagtcctt ttcggcaggt cgttcggccc |
| 61 | cgagtggagg gcaaacctgt gaacccaccc gagagcaaca aagcaggcga ctacagccac |
| 121 | gtgaagaccc tggaagctgt cctgaatttc aaatactctg gaggcccggg ccacactgaa |
| 181 | ggatattaca ggaatctctc cctggggctg catgtagaag tcgagccgtc tgta |

SEQ ID NO: 13 (NIBP211, mutF)

| | |
|---|---|
| 1 | ppesnkagdy shvktleavl nfkysggpgh tegyyrnlsl glhvevepsv fftrvstlpa |
| 61 | tstrqchlll dvfnstehel tvstrsseal ilhagecqrm aiqvdkfnfe sfpespgekg |
| 121 | qfanpkqlee errearglei hsklgicwri pslkrageas vegllnqlvl ehlqlaplqw |
| 181 | dvlvdgqpcd reavaacqvg dpvrlevrlt nrsprsvgpf altvvpfqdh qngvhnydlh |
| 241 | dtvsfvgsst fyldavqpsg qsaclgallf lytgdfflhi rfhedstske lppswfclps |
| 301 | vhvcaleaqa |

SEQ ID NO: 14 (Residues 1-120 of NIBP211)

| | |
|---|---|
| 1 | ppesnkagdy shvktleavl nfkysggpgh tegyyrnlsl glhvevepsv fftrvstlpa |
| 61 | tstrqchlll dvfnstehel tvstrsseal ilhagecqrm aiqvdkfnfe sfpespgekg |

SEQ ID NO: 15 (Residues 1-74 of NIBP211)

| | |
|---|---|
| 1 | ppesnkagdy shvktleavl nfkysggpgh tegyyrnlsl glhvevepsv fftrvstlpa |
| 61 | tstrqchlll dvfn |

SEQ ID NO: 16 (Residues 604-668 of NIBP1148)

| | |
|---|---|
| 1 | mvynpmpfel rvenmgllts gvefeslpaa lslpaesgly pvtlvgvpqt tgtitvngyh |
| 61 | ttvfg |

What is claimed:

1. A method for inhibiting NF-κB activation in a cell, comprising introducing into the cell a vector comprising a nucleic acid sequence encoding a NIK and IKK2 Binding Protein (NIBP) polypeptide having an amino acid sequence selected from the group consisting of SEQ ID NOs: 3 and 5-7, wherein the NIBP polypeptide is expressed in the cell.

2. The method of claim 1, wherein the cell is a cancer cell.

3. The method of claim 2, wherein the cancer cell is selected from the group consisting of breast, gut, liver, colorectal, cervix, prostate, lung and brain cancer cells.

4. The method of claim 1, wherein the cell has constitutive NF-κB activation.

5. The method of claim 1, wherein the cell has induced NF-κB activation.

6. The method of claim 5, wherein the NF-κB activation is induced by TNFα.

7. A method for reversing the cancerous phenotype of a cancer cell, comprising introducing into the cell a vector comprising a nucleic acid sequence encoding a NIK and IKK2 Binding Protein (NIBP) polypeptide having an amino acid sequence selected from the group consisting of SEQ ID NOs: 3-7 and 13-16, wherein the NIBP polypeptide is expressed in the cell.

8. The method of claim 7, wherein the cancer cell is selected from the group consisting of breast, gut, liver, colorectal, cervix, prostate, lung and brain cancer cells.

9. The method of claim 7, wherein reversing the cancerous phenotype comprises inhibiting cell proliferation and inducing cell death.

10. A method for modulating neuronal differentiation of a cell, comprising introducing into the cell a vector comprising a nucleic acid sequence encoding a NIK and IKK2 Binding Protein (NIBP) polypeptide, wherein the NIBP polypeptide is expressed in the cell, and the cell is selected from the group consisting of a neural stem cell (NSC), a neural progenitor cell (NPC), and a cell having disrupted NIBP expression.

11. A host cell comprising a vector comprising a nucleic acid sequence encoding a NIK and IKK2 Binding Protein (NIBP) polypeptide having an amino acid sequence selected from the group consisting of SEQ ID NOs: 1-7 and 13-16, wherein the cell is capable of expressing the NIBP polypeptide.


SEQUENCE LISTING (16 sequences)

SEQ ID NO: 1 - protein, 1246 aa, Homo sapiens
MVPAGDQDRAPHRGKPAQAGARTSRASRALRSWRRSQAARATVTHPRGGHDRGSHGGYREGHRGCRRDPQWASAGPPPLSFTEEVKFELRALKDWDFKMSVPDYMQCAEDHQTLLVVVQPVGIVSEENFFRIYKRICSVSQISVRDSQRVLYIRYRHHYPPENNEWGDFQTHRKVVGLITITDCFSAKDWPQTFEKFHVQKEIYGSTLYDSRLFVFGLQGEIVEQPRTDVAFYPNYEDCQTVEKRIEDFIESLFIVLESKRLDRATDKSGDKIPLLCVPFEKKDFVGLDTDSRHYKKRCQGRMRKHVGDLCLQAGMLQDSLVHYHMSVELLRSVNDFLWLGAALEGLCSASVIYHYPGGTGGKSGARRFQGSTLPAEAANRHRPGAQEVLIDPGALTTNGINPDTSTEIGRAKNCLSPEDIIDKYKEAISYYSKYKNAGVIELEACIKAVRVLAIQKRSMEASEFLQNAVYINLRQLSEEEKIQRYSILSELYELIGFHRKSAFFKRVAAMQCVAPSIAEPGWRACYKLLLETLPGYSLSLDPKDFSRGTHRGWAAVQMRLLHELVYASRRMGNPALSVRHLSFLLQTMLDFLSDQEKKDVAQSLENYTSKCPGTMEPIALPGGLTLPPVPFTKLPIVRHVKLLNLPASLRPHKMKSLLGQNVSTKSPFIYSPIIAHNRGEERNKKIDFQWVQGDVCEVQLMVYNPMPFELRVENMGLLTSGVEFESLPAALSLPAESGLYPVTLVGVPQTTGTITVNGYHTTVFGVFSDCLLDNLPGIKTSGSTVEVIPALPRLQISTSLPRSAHSLQPSSGDEISTNVSVQLYNGESQQLIIKLENIGMEPLEKLEVTSKVLTTKEKLYGDFLSWKLEETLAQFPLQPGKVATFTINIKVKLDFSCQENLLQDLSDDGISVSGFPLSSPFRQVVRPRVEGKPVNPPESNKAGDYSHVKTLEAVLNFKYSGGPGHTEGYYRNLSLGLHVEVEPSVFFTRVSTLPATSTRQCHLLLDVFNSTEHELTVSTRSSEALILHAGECQRMAIQVDKFNFESFPESPGEKGQFANPKQLEEERREARGLEIHSKLGICWRIPSLKRSGEASVEGLLNQLVLEHLQLAPLQWDVLVDGQPCDREAVAACQVGDPVRLEVRLTNRSPRSVGPFALTVVPFQDHQNGVHNYDLHDTVSFVGSSTFYLDAVQPSGQSACLGALLFLYTGDFFLHIRFHEDSTSKELPPSWFCLPSVHVCALEAQA

SEQ ID NO: 2 - protein, 1148 aa, Homo sapiens
MSVPDYMQCAEDHQTLLVVVQPVGIVSEENFFRIYKRICSVSQISVRDSQRVLYIRYRHHYPPENNEWGDFQTHRKVVGLITITDCFSAKDWPQTFEKFHVQKEIYGSTLYDSRLFVFGLQGEIVEQPRTDVAFYPNYEDCQTVEKRIEDFIESLFIVLESKRLDRATDKSGDKIPLLCVPFEKKDFVGLDTDSRHYKKRCQGRMRKHVGDLCLQAGMLQDSLVHYHMSVELLRSVNDFLWLGAALEGLCSASVIYHYPGGTGGKSGARRFQGSTLPAEAANRHRPGAQEVLIDPGALTTNGINPDTSTEIGRAKNCLSPEDIIDKYKEAISYYSKYKNAGVIELEACIKAVRVLAIQKRSMEASEFLQNAVYINLRQLSEEEKIQRYSILSELYELIGFHRKSAFFKRVAAMQCVAPSIAEPGWRACYKLLLETLPGYSLSLDPKDFSRGTHRGWAAVQMRLLHELVYASRRMGNPALSVRHLSFLLQTMLDFLSDQEKKDVAQSLENYTSKCPGTMEPIALPGGLTLPPVPFTKLPIVRHVKLLNLPASLRPHKMKSLLGQNVSTKSPFIYSPIIAHNRGEERNKKIDFQWVQGDVCEVQLMVYNPMPFELRVENMGLLTSGVEFESLPAALSLPAESGLYPVTLVGVPQTTGTITVNGYHTTVFGVFSDCLLDNLPGIKTSGSTVEVIPALPRLQISTSLPRSAHSLQPSSGDEISTNVSVQLYNGESQQLIIKLENIGMEPLEKLEVTSKVLTTKEKLYGDFLSWKLEETLAQFPLQPGKVATFTINIKVKLDFSCQENLLQDLSDDGISVSGFPLSSPFRQVVRPRVEGKPVNPPESNKAGDYSHVKTLEAVLNFKYSGGPGHTEGYYRNLSLGLHVEVEPSVFFTRVSTLPATSTRQCHLLLDVFNSTEHELTVSTRSSEALILHAGECQRMAIQVDKFNFESFPESPGEKGQFANPKQLEEERREARGLEIHSKLGICWRIPSLKRSGEASVEGLLNQLVLEHLQLAPLQWDVLVDGQPCDREAVAACQVGDPVRLEVRLTNRSPRSVGPFALTVVPFQDHQNGVHNYDLHDTVSFVGSSTFYLDAVQPSGQSACLGALLFLYTGDFFLHIRFHEDSTSKELPPSWFCLPSVHVCALEAQA

SEQ ID NO: 3 - protein, 224 aa, Homo sapiens
TVFGVFSDCLLDNLPGIKTSGSTVEVIPALPRLQISTSLPRSAHSLQPSSGDEISTNVSVQLYNGESQQLIIKLENIGMEPLEKLEVTSKVLTTKEKLYGDFLSWKLEETLAQFPLQPGKVATFTINIKVKLDFSCQENLLQDLSDDGISVSGFPLSSPFRQVVRPRVEGKPVNPPESNKAGDYSHVKTLEAVLNFKYSGGPGHTEGYYRNLSLGLHVEVEPSV

SEQ ID NO: 4 - protein, 146 aa, Homo sapiens
MEPLEKLEVTSKVLTTKEKLYGDFLSWKLEETLAQFPLQPGKVATFTINIKVKLDFSCQENLLQDLSDDGISVSGFPLSSPFRQVVRPRVEGKPVNPPESNKAGDYSHVKTLEAVLNFKYSGGPGHTEGYYRNLSLGLHVEVEPSV

SEQ ID NO: 5 - protein, 130 aa, Homo sapiens
TVFGVFSDCLLDNLPGIKTSGSTVEVIPALPRLQISTSLPRSAHSLQPSSGDEISTNVSVQLYNGESQQLIIKLENIGMEPLEKLEVTSKVLTTKEKLYGDFLSWKLEETLAQFPLQPGKVATFTINIKV

SEQ ID NO: 6 - protein, 52 aa, Homo sapiens
MEPLEKLEVTSKVLTTKEKLYGDFLSWKLEETLAQFPLQPGKVATFTINIKV

SEQ ID NO: 7 - protein, 78 aa, Homo sapiens
DGISVSGFPLSSPFRQVVRPRVEGKPVNPPESNKAGDYSHVKTLEAVLNFKYSGGPGHTEGYYRNLSLGLHVEVEPSV

SEQ ID NO: 8 - DNA, 672 bp, Homo sapiens
acggtcttcggtgtgttcagtgactgtttgctggataacctgccgggaataaaaaccagtggctccacagtggaagtcattcccgcgttgccaagactgcagatcagcacctctctgcccagatctgcacattcattgcaaccttcttctggtgatgaaatatctactaatgtatctgtccagctttacaatggagaaagtcagcaactaatcattaaattggaaaatattggaatggaaccattggagaaactggaggtcacctcgaaagttctcaccactaaagaaaaattgtatggcgacttcttgagctggaagctagaggaaacccttgcccagttccctttgcagcctgggaaggtggccacgttcacaatcaacatcaaagtgaagctggatttctcctgccaggagaatctcctgcaggatctcagtgatgatggaatcagtgtgagtggctttcccctgtccagtccttttcggcaggtcgttcggccccgagtggagggcaaacctgtgaacccacccgagagcaacaaagcaggcgactacagccacgtgaagaccctggaagctgtcctgaatttcaaatactctggaggcccgggccacactgaaggatattacaggaatctctccctggggctgcatgtagaagtcgagccgtctgta

SEQ ID NO: 9 - DNA, 438 bp, Homo sapiens
atggaaccattggagaaactggaggtcacctcgaaagttctcaccactaaagaaaaattgtatggcgacttcttgagctggaagctagaggaaacccttgcccagttccctttgcagcctgggaaggtggccacgttcacaatcaacatcaaagtgaagctggatttctcctgccaggagaatctcctgcaggatctcagtgatgatggaatcagtgtgagtggctttcccctgtccagtccttttcggcaggtcgttcggccccgagtggagggcaaacctgtgaacccacccgagagcaacaaagcaggcgactacagccacgtgaagaccctggaagctgtcctgaatttcaaatactctggaggcccgggccacactgaaggatattacaggaatctctccctggggctgcatgtagaagtcgagccgtctgta

SEQ ID NO: 10 - protein, 390 aa, Homo sapiens
ACGGTCTTCGGTGTGTTCAGTGACTGTTTGCTGGATAACCTGCCGGGAATAAAAACCAGTGGCTCCACAGTGGAAGTCATTCCCGCGTTGCCAAGACTGCAGATCAGCACCTCTCTGCCCAGATCTGCACATTCATTGCAACCTTCTTCTGGTGATGAAATATCTACTAATGTATCTGTCCAGCTTTACAATGGAGAAAGTCAGCAACTAATCATTAAATTGGAAAATATTGGAATGGAACCATTGGAGAAACTGGAGGTCACCTCGAAAGTTCTCACCACTAAAGAAAAATTGTATGGCGACTTCTTGAGCTGGAAGCTAGAGGAAACCCTTGCCCAGTTCCCTTTGCAGCCTGGGAAGGTGGCCACGTTCACAATCAACATCAAAGTG

SEQ ID NO: 11 - DNA, 200 bp, Homo sapiens
atggagaaagtcagcaactaatcattaaattggaaaatattggaatggaaccattggagaaactggaggtcacctcgaaagttctcaccactaaagaaaaattgtatggcgacttcttgagctggaagctagaggaaacccttgcccagttccctttgcagcctgggaaggtggccacgttcacaatcaacatcaaagtg

SEQ ID NO: 12 - DNA, 234 bp, Homo sapiens
gatggaatcagtgtgagtggctttcccctgtccagtccttttcggcaggtcgttcggccccgagtggagggcaaacctgtgaacccacccgagagcaacaaagcaggcgactacagccacgtgaagaccctggaagctgtcctgaatttcaaatactctggaggcccgggccacactgaaggatattacaggaatctctccctggggctgcatgtagaagtcgagccgtctgta

SEQ ID NO: 13 - protein, 310 aa, Homo sapiens
PPESNKAGDYSHVKTLEAVLNFKYSGGPGHTEGYYRNLSLGLHVEVEPSVFFTRVSTLPATSTRQCHLLLDVFNSTEHELTVSTRSSEALILHAGECQRMAIQVDKFNFESFPESPGEKGQFANPKQLEEERREARGLEIHSKLGICWRIPSLKRSGEASVEGLLNQLVLEHLQLAPLQWDVLVDGQPCDREAVAACQVGDPVRLEVRLTNRSPRSVGPFALTVVPFQDHQNGVHNYDLHDTVSFVGSSTFYLDAVQPSGQSACLGALLFLYTGDFFLHIRFHEDSTSKELPPSWFCLPSVHVCALEAQA

SEQ ID NO: 14 - protein, 120 aa, Homo sapiens
PPESNKAGDYSHVKTLEAVLNFKYSGGPGHTEGYYRNLSLGLHVEVEPSVFFTRVSTLPATSTRQCHLLLDVFNSTEHELTVSTRSSEALILHAGECQRMAIQVDKFNFESFPESPGEKG

SEQ ID NO: 15 - protein, 74 aa, Homo sapiens
PPESNKAGDYSHVKTLEAVLNFKYSGGPGHTEGYYRNLSLGLHVEVEPSVFFTRVSTLPATSTRQCHLLLDVFN

SEQ ID NO: 16 - protein, 65 aa, Homo sapiens
MVYNPMPFELRVENMGLLTSGVEFESLPAALSLPAESGLYPVTLVGVPQTTGTITVNGYHTTVFG